SEQUENCE LISTING (13 sequences)

SEQ ID NO: 1 - protein, 980 aa, Artificial Sequence
  Artificial protein including a signal sequence, receptor binding domains, linker, a tag, fusion protein transmembrane, 2A cleavable peptide, CD5 signal sequence, surfactant protein D tetramerization domain, FLAG tag, and CD40L
MITIVATTVILLLPLSLCQIDQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSGGGGSGGGGSGGGGSHHHHHHNCIPTTCTCDGIDNRINQAPDQGIRVITHKECQVIGINGMLFRPNKEGTLATYTYDDIVLNNSVALDPIDISMELNKAKLELEESKEWIKKSNQKLDSVGGWYQSNITITIIIVMIMILFIINMIIIIIIFKQFKIRNRHPIDKSNEPYILTRKQGSGATNFSLLKQAGDVEENPGPMAMGSLQPLATLYLLGMLVASCLGLQLDPAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGDYKDDDDKLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - protein, 21 aa, Artificial Sequence
  F signal protein
MITIVATTVILLLPLSLCQID

SEQ ID NO: 3 - protein, 271 aa, Coronavirus
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 4 - protein, 15 aa, Artificial Sequence
  Linker sequence
GGGGSGGGGSGGGGS

SEQ ID NO: 5 - protein, 6 aa, Artificial Sequence
  His tag
HHHHHH

SEQ ID NO: 6 - protein, 155 aa, Coronavirus
NCIPTTCTCDGIDNRINQAPDQGIRVITHKECQVIGINGMLFRPNKEGTLATYTYDDIVLNNSVALDPIDISMELNKAKLELEESKEWIKKSNQKLDSVGGWYQSNITITIIIVMIMILFIINMIIIIIIFKQFKIRNRHPIDKSNEPYILTRKQ

SEQ ID NO: 7 - protein, 22 aa, Artificial Sequence
  2A Autocleavable Peptide
GSGATNFSLLKQAGDVEENPGP

SEQ ID NO: 8 - protein, 29 aa, Artificial Sequence
  CD5 Signal Sequence
MAMGSLQPLATLYLLGMLVASCLGLQLDP

SEQ ID NO: 9 - protein, 237 aa, Coronavirus
AEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNG

SEQ ID NO: 10 - protein, 8 aa, Artificial Sequence
  FLAG tag
DYKDDDDK

SEQ ID NO: 11 - protein, 216 aa, Artificial Sequence
  CD40L
LHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 12 - protein, 782 aa, Artificial Sequence
  Protein including CD5 signal, coronavirus receptor binding domains, linker, His tag, SPD, FLAG tag, and CD40L functional domain
MAMGSLQPLATLYLLGMLVASCLGLQLDPQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSGGGGSGGGGSGGGGSHHHHHHAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGDYKDDDDKLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 13 - DNA, 15962 bp, Artificial Sequence
  BPIV3 Backbone
gtcgactaatacgactcactatagggagattggtctgatgagtccgtgaggacgaaacggagtctagactccgtcaccaaacaaggggagagacttgtttggaaatatgaatttaaatagaaattaacttaggattaaagaactttaccgaaaggtaagggagaagaaatccttaaattgtcatcatgttgagtctgtttgatacattcagtgcacgcaggcaggaaaacataaccaaatcagctggtggagctgttattcctgggcaaaagaacacagtctctatatttgcccttggtccctcaataacagatgacaatgataaaatgacattagctctcctgttcttatctcactcactagataatgagaagcagcatgcacaaagagctggatttttagtctccttgctatccatggcttatgctaacccagaattatacctaacatcaaatggtaataatgcagatgtcaaatatgttatctatatgatagaaaaagactcagggagacaaaaatacggtggctttgttgttaagactagggaaatggtttatgagaagacaactgattggatgtttggaagtgatcttgagtatgatcaagataatatgttacagaatggtaggagcacctccacgattgaagatcttgttcacacatttgggtatccagcatgccttggagctcttataatccagatttggataatacttgttaaagctataaccagtatctcaggattgagaaaaggtttttttactcggttagaggcatttagacaggatgggacagtcaaatccagtctggtattgagtggagatgcagtagagcagatcggatcgatcatgaggtctcagcaaagtttggttacgcttatggtcgagacactgataacaatgaacacaggaaggaatgatctaacaacgatagagaagaacatacaaattgtaggaaactacataagagatgcaggccttgcttcattctttaatacaatcagatatggcattgaaaccagaatggcagctctaacattgtctactcttagaccagacatcaacagactcaaagcattaatagagttatacctatcaaaggggccacgtgccccttttatatgtattttaagggatcctattcatggcgagtttgcaccaggcaattatcctgctctctggagttatgcaatgggtgtagcagttgtccagaacaaagctatgcaacagtatgtaacaggacggtcctatctagatattgagatgttccaactaggtcaagcagtggcacgtgacgccgagtcacaaatgaattcaatattggatgaagaattggggattacacaagaggccaagcagagtttgaagaaacacatgaagaaaatcagtagttcggacacaactttctacaaacctacagggggaacagccatagaaatggcaatagacgaggagatagaacaaccagaatccagaggagaccaggatcaaactgatgatcttcagtcatctataatcccctatgcatgggcagacgaggcaggcaacaaaaaccaagcagggtcaaccgcagacgttgacagcctcggagttgagcaaagaaacatcagagatagattgaacaaaaggctcaacgaaaagagaagacagaatgaatcaaaatcagctgacataacaaataatgctgaccagactgagatagatgacctgttcagcgcattcggaagtagctaattaaatagtggtgattattaccatttgcaacaaataagaaaaacttaggattaaagagattacccagatttcacaagcacaatcaaaccagcacccagagacaacacggacagagcagaagaggcaccaggggaagaaccaacaaacaggaaagaggaaggaacaacagaaaacaggggcaaacaccaaagagcaaaggaacaccaccaacgaacataccagcaaccccacaacagaacacaagaaaaaagaatagaaaacgaagagcaccaaagaccacgcgtctcgagaccggaaatgtagcaccaaacatcaagggtataaaaaacttaggattaacggagattatccaatcctgacacagcagaccaagaccagactccatctacaacccaaccgatcgagaattcatggaagacaatgttcaaaacaatcaaatcatggattcttgggaagagagatcaggagacaagtcatccgacatctcatcagccctcgacatcattgaattcatactcagcaccgatccccaagaaagcgcggtcagcgacaatgagaacggcgcaggggtcacaaggctcagcacaacaatccaccggcccgacccgaaatcaacaaagacaggcgaggaaaatagtggaccaactaacgaagatcgacagcttgggacatcatacaaatataccgcagaaacaaaagatagagttgttaatcaggaagctgtacagagaagaaataggagaggaagcagcccagatagtagaactgagactatggtcattagaagaatcaccggaggcagctcagatcctgacaatggaaccgaaatccaggaaaatcttgattacaatgaaactagagaggtggataagaaccctactaaggggaaagtgcgacaacttgaaaatgttccagtcaaggtaccaggaagtgatgccatacctccagcaaaatcagatggagacagtgatgatggaggaggcctggaatctatcagtacacctaatccaagacatactagcctagtgaccacagcaacaccagatgacgaagaagagcttatctcgaaaaacaaaaggtcaaaaaagcaccagctaacaaaccagagaagtaacaaagaaattaaaaaagggggagaggaggagatttggaagcaagcagatatcgaccatcagtcatccagattggactacgaaccagacttcaaaggatcgaagaggaaccagaggatcctcaagaccagcacagatgcaggggaaccaacaagaatacagaatgggtcccaaaggaagagaatcacatcctggaacatcctcaacaacaagagcagcaatcgagaggaacagacagtcagaaatccccaacgatcagcacatggacagaagcaaacaatggtatcagacagatcagctccagaacaacaagtcaaagcacagaagacgaacggagaggaaggaaaggacacagaagagagcactcaatttacagaaagggcgattacattattacagagtcttggtgtaatccaatctgcagcaaaattagacctgtaccaagacaagagagttgtatgtgtggcgaatgtcctaaacaatgcagatactgcatcgaagatcgattttttagcgggtctgatgattggagtatcgatggatcaagacaacagacttaatcagattcaaaatgagataatagatttaaagactgatctcaaaaagatggatgaatcacacagaaggttgattgagaatcaaaaagaacaactatcattaatcacatcattaatttcgaatcttaagatcatgacagagagaggaggaaagaaagatcaggttgagacaagtggaagaacatccatgataaaaacaaaaatgaaagaagagaagataaggaaagttaagttcgatcctcttatggaaacacaaggcatcgaaaagaatgttcctgatttatacagatcgatagagaaggcaccagaaagtgacgtgcagatcaaatcagaaattagtaggttaagtgatgaatcgaatgcaactagactaataccaagaagaataagcagcacaatgaggtcattaatcataatcatcaataacagcagcctgtcatcaaaggcaaaacagtcatacataaatgaactcaagctctgcaagagtgatgaagaagtatctgaattgatggatatgtttaatgaagacattagctcccaatagacagccaaacgacaatcaccaccacagaacacactgacacacatcagcaaaccaaagactatccccaactaccaagtcaatcaacacaccacagtagttcccagacacaagccaatcaagaaaaacttaggacaaaagagccaatcagtccctcaacaaaatgagcatcacaaactccgcaatatatacatttccagaatcttcattttctgaaagtggcaatatagagccactaccactaaaggtcaacgaacaaaggaaggcaattcctcacatcagagttgttaaaataggtgacccacccaaacatggttcaagatatcttgatgtatttctactgggtttttttgagatggagcggtcaaaggacagatatgggagtataagtgatctggatgatgatccaagttataaagtttgtggttctggatctctaccacttgggctagcaagatacactgggaatgatcaggaactcctacaagccgcaattaagttagacatagaagtgagaagaaccgtcaaagccacggagatgatagtctacaccgtacagaacattaaacctgaactgtacccatggtcaagcaggttaagaaaagggatgttatttgacgccaacaaagttgcgcttgcacctcaatgtcttccactcgatagagggataaaattcagagtaatatttgtgaattgtacagcaattggatcaataacactctttaaaattcccaaatctatggctttgctatcattgccaaacactatatcaataaatttacaggtacacataaaaacaggaattcaaactgactctaagggtgttgtccagatcttagatgaaaaaggtgagaaatcgttaaactttatggtccatcttggattaattaaacggaaggtaggtcgaatgtattcagtagagtattgcaaacaaaagattgagaagatgagactactattttcattgggattagttggaggaatcagctttcatgtcaatgcaactgggtccatttcaaaaacgttggcgagtcaactagcgttcaaaagggaaatctgttatcctctcatggatttaaatccacacctaaatctggttatatgggcatcatcaattgaaattacaagagtggatgcaatccttcaaccttcattgcccggtgagttcagatattacccaaacataatagcaaaaggagtcgggaaaatcaagcagtaacaccgacagcctggacaagcaacattataaatcagatcatttaaaaaaccaaaatcaaaaacttaggattaaagagattacccagatttcacaagcacaatcaaaccagcacccagagacaacacggacagagcagaagaggcaccaggggaagaaccaacaaacaggaaagaggaaggaacaacagaaaacaggggcaaacaccaaagagcaaaggaacaccaccaacgaacataccagcaaccccacaacagaacacaagaaaaaagaatagaaaacgaagagcaccaaagatgaccaccatagttgcaacaacagttatattattattacctttatcactatgtcagatagatataacaaagttgcaacgagtaggggtgctagtcaacaatccaaaaggcatgaagatttcacaaaattttgaaacaagatacttaatattaagtttaataccaaagatagagaactcacaatcatgtggagaccaacaaataaatcaatataagaaattattagatagattaattattcctttatatgatgggttaaaattacagaaagatgtaatcgtagtaagacatgagtctaataacagtactagttccaggaagaagcggttcttcggagaaataattgggacaattgcgatagggatagccacatcagctcaaataactgcagcagttgcccttgttgaagccaaacaggcaagagcagatatagacaagctcaaggaagccatcagagatacaaacaaggcagtacagtcaattcaaagctcagtaggaaatctaattgttgcggttaagtcagttcaagattacgtcaataatgagattgtaccatcaatagcaagattaggctgtgaagcagcaggattacaactcggaattgcattgacacaacattactcagagttaacaaatatatttggtgataacataggaacactgaaggagaagggaataaaattacaagggatagcatcattatatcgcacaaacataacagagatattcactacttcaacagttgatcaatatgacatttatgatctattgttcactgagtcaatcaaaatgagagttatagatgtagacttaaatgactattcaattacactacaagttagactacctttattaacaaaattatcaaacactcagatttatagagtggattctatatcatataatatccaaggtaaggaatggtacattcctcttcccaatcacatcatgacaaagggggcatttttgggtggtgctgatataaaagagtgtatagaggcattcagcagttacatatgtccctctgacccaggttacatattaaatcgtgagatagaaaactgtctatcagggaatatcacacagtgtccgaaaaccgttgttacatcagacatagtaccaagatatgcatttgttaatggcggagtaattgccaattgtataccaaccacttgtacatgtgatgggattgacaataggatcaaccaggcacccgatcaagggataagggtaataacacacaaagaatgtcaagtgataggtataaacgggatgttattcagacctaacaaagaagggactttggcaacttatacatatgatgacattgttttaaacaattctgtcgcgcttgacccaattgacatatcaatggaactcaacaaagctaaattggaattagaagaatcaaaagaatggataaagaagtcaaatcaaaaattggactctgttggaggttggtatcagtcaaatataacaattactataataatagtgatgataatgattctgtttataatcaacatgataatcattataataatctttaaacaattcaagattcgcaatagacacccaattgacaagagcaatgaaccctatatactgactaggaaacagtagggaaatgtagcaccaaacatcaagggtataaaaaacttaggaacaaagcagtcccactcagagacaaagaacagacgacacggaatcccaacctcaaaaatggaatattggaggcacacaaacaacgccaaaaacaccaacactgaattccaaacagagacaactaggcgtaataataaagttacaaataccatgatgtccaccttcggggcaatactctcaactatattgttgacagtgttcataatgatactggtaggtttgattcaagaaagcaaccacaacaaaatagcatcacaacaaatgagaagagaattcgcagaaattgagaaaaaaatccagcaagctgcagatgaaattggtacatcaatacaatcgggaataaacacaagactcctaactatccagagtcatgtccaaaattatattccattatctttgacacaacaaatgtcggatcttagaaaatttatcaatgaattagcaaataagagagatcaacaggaggtaccaatacagagaatgactcatgacagcgggattgaacccctaaaccctgacaaattctggagatgtacctctggcaatccatccttgacaagtagcccaaagataagactaataccaggtcctagtctattagcagcatctaccactgtaaatggttgtataaggatcccgtccttcgtaatcaaccatctaatatatgcttacacttccaaccttattgttcaaggttgccaggatatagggaagtcttatcaggtattacaaatcgggatcatcactataaattcagacctggtacctgacttaaaccctagagttacacatacttttaatattgatgataatagaaagtcctgttctttggcactactaaatacagatgtatatcagttgtgctcaactccgaaagttgacgaaagatcagactatgcatcaacagggattgaagacattgtacttgatattattactaacaatggattaatcatcacaacaagatttaccaatgataatataacatttgacaaaccatatgcagcactgtacccatcagtaggaccggggatttattataaaggtaaggttatttttctcggatatggtggattagaacatgcagagaatggtgatgtaatatgcaatttaactggttgtccaggcaaaacacagagagactgtaatcaagcttcctacagcccatggttttcagataggagaatggtgaactctattattgttgttaataaaggtgtggacgcaactttcaatttaagagtatggactatcccaatgagccaaaactattggggatcagaagggagattacttttattaggcaataagatatacatatatactagatctacaagttggcatagtaaactccaattagggacaattgatattaccaattacagtgatatcaggatcaattggacctggcatgatgcattatcgagaccaggaaatgatgattgtccgtggggtcactcatgcccggatgggtgtataacaggggtttatactgatgcatacccgttaaacccatcaggaagtgttgtgtcatcagttattcttgattctcgaaaatccagggaaaatccgatcatcacttacgcaacagacacacgtagaataaatgaactggctatatataacagaacacttccagctgcatacacaacaacaaattgcattatgcattatgataagggatactgcttccacattgtcgagataaatcataggagcctaaatacattccaacctatgttattcaaaacagaaattccaaagaactgtagttgagttgactctcccatgttaattatcaaacacggaagacaagcatcaacaggtaggggatccaaaaaatgagatgacaaatagaggaaaaataaaaaacttaggagaaaagtttgtcgggaaaatggacaccgaattcagcggtggcacaacatctgatattttatatccagaatgtcatctcaattctcctatagtgaaagggaaaatagcacaattacacactataatgagtttgcctcaaccttatgatatggatgatgactcaatactagttataactaggcagaagattaggctgaataaattagataaaagacaacgatcaatcaggaaattgaaatctgtcctaatggaaagagtaattgacttaggtaaatataactttattaggtatccagaaatgtctagtgaaatgttccaattgtgtatacctggaattaatagcagaataaatgaattattaagtaaagcacataggacttataatcaaatgactgatggattaagggatttatgggttactatactatcaaaattggcatcaaaaaatgatggaagtaattataatattaacgaagatatcagtaacatatctagtgttcacatgacctatcaatcagatagatggtataatccattcaagacatggtttaccattaaatacgatatgaggaggctacaaaaagccaaaaatgaggttacatgtgataagtacaaggattacaatttattagaagatcaaaggaatgtattactgatacatacggagcttgtcctcatactagacaaacaaaattataaaggatatataataacccctgaattggtattaatgtattgtgacgtagttgaaggaagatggaacattagctcgtgtgcaaaactagaccctaaattacattcaatgtattacaaaggtaacaacttgtgggaaacaatagatgggttgttcccagaattaggagagaaaacttttgatataatatcattgttagaaccactagctttgtcactcatacaaactcatgatccagttaagcaattacgtggagcctttttgaatcatgtattatctgaaatggagtcaatattagacaccaggtacaagaccgaagaaacacctgaattggactacatagacaaaattctagatgtattcaaagaatcaacaatagatgaaatagcagagattttctcttttttccgaacttttggacatcctccattagaagctagtatagcagcagaaaaagtcaggaaatatatgtatactgagaaaagcctaaattttgatactattaataaatgtcatgctattttttgtacaatcattataaatgggtatagagaaagacacggaggacaatggcctccaattatattgcctgctcatgcacatgaattcattataaatgcttatggatcaaattctgctatatcatacgaaaatgctgttgattactataggagctttataggaattaagttcaacaaatttatagaacctcaattagatgaagacttaactatttatatgaaagataaagcattatctccaaaaaaatcaaactgggacaccgtatatccatcctcaaacttattgtatcatactagtacatcccatgattcaagaaggttagttgaggtttttatagcagatagtaaatttgaccctcatcaggtattagattatgtggagtcaggagaatggttgaatgatcctgagttcaatatctcatatagtctaaaagagaaggagataaagcaagaaggtaggctttttgcaaagatgacatataaaatgagagctgcgcaagttttgtcagaaacgttattagcaaacaatatagggaaattctttcaggagaatggaatggtcaaaggagagattgagctacttaaaagattgacaacaatatctatgtctggagtcccacgatacaatgaagtatacaataatcccaaaagtcatacagatgagatcaaagcttacaacgcgattagtaattctaatttatcttctaaccaaaaatcaagaagattcgaattcaagtcaacagacatatacaatgacggatatgaaactgtaagttgttttttaacaacagatctaaagaaatattgtttgaattggagatatgaatcaacagctctatttggtgatacttgcaatcagattttcgggttaaaaaagttattcaattggttgcatcctcgccttgaaaagagtacaatatatgttggagatccctattgcccgcccttagatatagaacacttacaacttgatgatcaccctgattcagggttttacgttcataatcctagaggaggaatagaaggattttgtcaaaaattgtggacacttatatctattagcgcaatacacctagcggctgtcaaaataggtgttagagtgactgcaatggtgcaaggagacaatcaagccattgctgtgactacaagggtgccaaataactatgattacaggatcaagaaggaaattgtttacaaggatgtggttaagttttttgattctttaagagaggttatggatgatctgggtcatgaacttaaattaaatgaaacaataataagcagtaaaatgtttatatacagcaaaagaatatattatgatggaagaattctccctcaagctttaaaagcattatctagatgtgtcttttggtctgaaaccattatagatgaaacaagatcggcatcttctaatttagcaacatcatttgcaaaagctattgagaatggttattcacctgtgttgggatatgtgtgttcaatattcaaaaatatccaacaattgtatatagcactcggaatgaatattaatcctactataactcagaatatcagagatcaatattttaggaacacccattggatgcaatatgcttctttaatacctgcaagtgttggagggtttaattatatggccatgtctaggtgttttgtaagaaacattggggaccccacggttgccgcattggcggatattaaaagatttataaaagcaaatctgttggatcgtggtgttctttatcgaattatgaatcaagaaccaggtgaatcttcgtttctagactgggcttctgatccgtactcatgtaacttaccacaatcccaaaatataacaactatgattaaaaatataactgcaagaaatgtactacaagattcaccaaatccattattatcaggattatttacaagcacgatgatagaagaggatgaagaactcgctgagtttttgatggataggagaataattcttccaagagttgcacatgatattttagataattctcttactggaatcagaaatgctatagcaggaatgctggacacaacaaaatcgttaattcgagtagggataaatcgaggagggttaacttatagtttgttaagaaaggtaagtaactatgatcttgtacaatatgagacacttagtagaaccttaagattaattgttagtgataagataaagtatgaagacatgtgctcggtagatctagctatctcgttaagacaaaaaatgtggatgcatttatcaggaggaagaatgataaatgggcttgaaacacctgatccattagaactattgtctggagtaataataacagggtctgagcactgcaggatatgttattcaactgaaggagaaagcccatatacttggatgtatttacctggtaatctgagtataggatcagccgaaaaagggattgcatcattaagagtcccttattttgggtcggtaacagatgagagatctgaggcacaactggggtacattaagaatttaagcaaaccagcaagagctgctataagaatagcaatgatctatacttgggcatttggcaatgatgagatatcctggatggaggcatcacagattgcacagacacgtgcaaatttcacgttagatagcttaaagattctgacaccagtgactacatcaacaaatttatcacacagattaaaggataccgctacccagatgaaattttctagtacatcacttatcagagtaagcagatttatcacaatatcgaatgataatatgtctatcaaagaagctaacgaaacaaaagatacaaatcttatttatcaacaggtaatgttaacagggttaagtatatttgaatatttatttagattagaagaaaatacagggcacaatcctatggttatgcatctacatatagaagacgggtgttgtattaaagaaagttacaatgatgatcatatcaatccagagtccacattggagttgattaggtatcctgaaagtaatgagtttatatatgacaatgaccctctcagagatgtagacctgacaaaactaatgattataagagatcactcctatactattgatatgaattattgggatgacacagatattatacacgcagtatcaatatgtacagcaatcacaatagcagacaccatgtcacagcttgaccgggacaatcttaaggagctaatagttattgcaaatgatgatgatataaatagtcttatcactgaattcctaacccttgacatattagtatttcttaaaacattcggaggattacttgttaatcaattcgcatatactctatatgggttgaaaatagaaggaagggatcctatctgggactatataatgaggacactaaaagacacctcacactcaatattaaaagttttgtcaaatgcattatcccatccaaaggtttttaaaaggttctgggattatggagttttaaatcctatctatgggcctaacactgctagtcaagaccaaatcaagcttgctctttctgtttgtgagtattctcttgatttatttatgagagaatggctgaatggtgcatcgcttgaaatctatatttgcgacagcgatatggaaatagcaaatgacaggagacaagcatttgtttcgagacatctcgcgtttgtgtgttgtttagcagaaatagcatcatttggaccgaatttattaaacttgacgtatttggaaaggttagaagaattaaagcggtatttagatttaaacattaaagaagatccaactcttaaatacgtacaagtatcaggactattaatcaaatcattcccttcaactgttacatatgtaagaaagacagcaattaaatatctaagaattcgtggaattgacccacctaacatacttgaggattgggatcccatagaagatgagaatatattagataatattgttaggactgtgaatgataattgtaacgataatccaaggaaatgtaagagtaattatttttggggtttagctcttaaaaattatcaagttgtgaggataagatctataaccagtgatcctgaagtttatgagacctcagatgatattgaacatggattgacacttcctgagggaggaagttatttatcacaccagctgcgattatttggagtgaatagtacaagttgtatcaaagcccttgagctatcgcagattctaatgaaagaagttaaaaaagataaggatagacttttcctaggagagggagcaggtgctatgctagcatgttatgatgccacacttggtcccgcaataaattattacaattctggtctaaatattacagatgtaattggtcaaagagaattaaaaatctttccatcagaagtttcattagtaggtaagaaactaggtaatgtgacacagatcctcaatcgtgtgaggatattatttaacgggaaccctaactcaacatggataggaaatatggaatgcgaaagtttaatatggagtgaattacatgacaaatcaattggtttagtacattgtgatatggaaggagcaataggtaaatcagaggaaactgtcctacatgaacactatagtattattagaatcacatatttagttggagacgatgatgttatcttggtatccaaaatcataccaaccatcactccaaattggtctaaaatacttcatttatataaattgtattggaaggatgtgagaataatatcccttaagacatccaaccccgcctcgacagagctgtatctaatttctaaggaagcatattgtactgtgatggagcctagtaatcttgtgttatcaaaactcagaagaatatcatcaatagaagagaataatctactaaagtggataatcttatctaaaaagaagaataatgaatggttgcagcatgaaattaaagaaggagaaagagactatggagtaatgagaccctatcacacagcactgcaaatattcggattccagattaatctaaaccatctagcaaaagagtttctatcaactcctgatttaaccaatattaacattgtgatacaaagtttcataagaacagtcaaagatgttatattcgaatgggtaaacataactcatgacaacaaaagacataaattaggagggaaatataatttgtttccgctcaaaaatagaggaaagttaaaattactttcacggagattagtgttgagctggatatcattatctctatcgaccagattactgacgggtcgtttcccggatgagaagtttgagggtagagcacagaccgggtttgtgtcattagctgacactgatctcgaacctctaaaattactatccaaaactcttgtcagagattataaagaacacataggattgatatcatattggtttctaaccaaagagatcaaaatactgatgaaacttataggaggagttaaattattggggatccctaaacaatataaagaacttgaacaccaatcatatccagattatgaatatgataatgaatttgatattgattaatgtataaatccagtaaatagtgtacttaatccctatctgtatatattaccaaccaaaaacaataagaaaaacatatgatatatatataccaaacagagtttttctcttgtttggtggccggcatggtcccagcctcctcgctggcgccggctgggcaacattccgaggggaccgtcccctcggtaatggcgaatgggacgctagttattgctcagcgggctagttattgctcagcgggtcgac

(19) United States
(12) Patent Application Publication
(10) Pub. No.: US 2023/0167159 A1
(43) Pub. Date: Jun. 1, 2023

(54) SARS-CORONAVIRUS 2 (SARS-COV-2) SUBUNIT VACCINE CANDIDATES

(71) Applicant: Kansas State University Research Foundation, Manhattan, KS (US)

(72) Inventors: Waithaka Mwangi, Manhattan, KS (US); Huldah Sang, Manhattan, KS (US)

(21) Appl. No.: 17/906,825

(22) PCT Filed: Mar. 22, 2021
(86) PCT No.: PCT/US2021/023529

§ 371 Date: Sep. 20, 2022

Related U.S. Application Data
(60) Provisional application No. 62/992,669, filed on Mar. 20, 2020.

(51) Int. Cl. C07K 14/165; C12N 15/86

(52) U.S. Cl. CPC C07K 14/165; C12N 15/86; C12N 2770/20034; A61K 2039/53

(57) ABSTRACT

A new species of coronavirus, SARS-CoV-2, is the cause of a worldwide pandemic and has resulted in hundreds of thousands of deaths. The present disclosure provides immunological compositions and methods related to the production and administration of such compositions to reduce the severity of, incidence of and transmissibility of SARS-CoV-2.

This application relates to and claims priority to U.S. Provisional Patent Application No. 62/992,669, which was filed on Mar. 20, 2020 and is incorporated herein by reference in its entirety.

### SEQUENCE LISTING

This application contains a sequence listing in paper format and in computer readable format, the teachings and content of which are hereby incorporated by reference.

### BACKGROUND OF THE DISCLOSURE

The coronavirus disease 2019 (COVID-19), caused by the novel pathogenic SARS-coronavirus 2 (SARS-CoV-2) that broke out in Wuhan China, is rapidly spreading globally. The SARS-CoV-2 is a novel virus that belongs to the genus betacoronavirus and it is related to Bat and Severe Acute Respiratory Syndrome coronavirus (SARS-CoV). Similar to other coronaviruses, the SARS-CoV-2 utilizes the spike (S) protein to infect host cells by binding to human angiotensin-converting enzyme 2 (ACE2), but with much higher affinity. The S protein, which exists as a trimer displayed on virus surface, contains ACE2 receptor binding domains [RBD] and this knowledge is relevant to vaccine development, since antibodies capable of blocking this interaction can neutralize infection. Notably, the SARS-CoV-2 RBD [S protein amino acid 319-591] alone has been shown to be sufficient for binding to human ACE2 receptor, suggesting that this region is a good target for inducing neutralizing antibodies. Consequently, there is an urgent need to develop safe and effective vaccines to protect the human population. What is needed are compositions that are effective at reducing the incidence of, reducing the severity of, and/or reducing the transmissibility, of the pathogenic SARS-coronavirus 2 (SARS-CoV-2).

### SUMMARY OF THE DISCLOSURE

The present disclosure addresses the problems inherent in the art and provides three different types of vaccines or immunogenic compositions effective for reducing the incidence of, reducing the severity of, and/or reducing the transmissibility, of the pathogenic SARS-CoV-2. The first vaccine or immunogenic composition is a recombinant protein expressed in mammalian cells, the second utilizes a mutant Bovine Parainfluenza-3 Virus genotype C (BPI3Vc) replicon vector for mucosal and parenteral antigen delivery, and the third is a nucleic acid-based immunogenic composition or vaccine.

In some forms, Recombinant attenuated Bovine Parainfluenza 3 Virus, genotype C [BPI3Vc] encoding the SARS-CoV-2 RBD [S protein amino acid 319-591 or 321-591]. Preferably, sequences used for purposes described herein will have at least 90, 91, 92, 93, 94, 95, 96, 97, 98, 99, 99.5, 99.9 or even 100% sequence identity with the RBD sequence depicted in FIG. 1. The RBD is designed for display on the surface of the infected cells: this is mediated by the signal sequence and the transmembrane domain of the BPI3Vc Fusion [F] protein [FIG. 1]. It is understood that other signal sequences could be used, but the sequence provided below in FIG. 1 is preferred. Alternatively, sequences having at least 90, 91, 92, 93, 94, 95, 96, 97, 98, 99, 99.5, 99.9 or even 100% sequence identity with the F signal protein sequence depicted in FIG. 1 could also be utilized for purposes of the present disclosure. Surface display of the RBD is expected to mimic presentation of the RBD by the S protein on the virus surface and we hypothesize that the RBD will be amenable for optimal B cell recognition and response. In addition, the construct is designed for co-expression of the human surfactant protein D [SPD]tetramerization domain [fused to human CD40L [FIG. 1]. A codon-optimized synthetic gene encoding the chimeric polypeptide, designated _{FLP}RBD_{FTM} 2A-_{CD5}SPD-CD40L, was outsourced from GenScript [paid service] and the gene was cloned into pCDNA3.1 (+) to generate a recombinant construct, designated p_{FLP}RBD_{FTM}-2A-_{CD5}SPD-CD40L. The expression cassette from this plasmid construct was subcloned into BPI3Vc vector and the resultant recombinant virus construct is designated BPI3Vc-_{FLP}RBD_{FTM}-2A-_{CD5}SPD-CD40L [FIG. 2A]. Preliminary data shows that the encoded protein is expressed on the surface of human embryonic kidney cells (HEK) 293 cells transfected with the p_{FLP}RBD_{FTM}-2A-_{CD5}SPD-CD40L plasmid construct as judged by immunocytometric analysis using anti-His-tag monoclonal antibody [mAb] and more importantly by anti-corona virus S protein mAb [FIG. 2]. The rescued BPI3Vc-_{FLP}RBD_{FTM}-2A-_{CD5}SPD-CD40L virus is expressing the antigen in a similar manner.

The vaccine or immunogenic composition preferably comprises the mutant BPI3Vc replicon vector as an antigen delivery vector. Advantageously, the mutant BPI3Vc vector can also be used as a platform for other broadly protective vaccines via antigen delivery.

U.S. BPI3Vc isolates include KJ647285.1 Bovine parainfluenza 3 virus isolate TVMDL16, and KJ647287.1 Bovine parainfluenza 3 virus isolate TVMDL20. In China, BPI3Vc isolates include HQ530153.1 and KT071671.1. A BPI3Vc isolate in South Korea is JX969001.1 and a BPI3Vc isolate in Japan is LC000638.1. In some forms, the vector of the present disclosure will have at least 80% homology to one or more of these BPI3Vc isolates. More preferably, the homology will be at least 81, 82, 83, 84, 85, 86, 87, 88, 89, 90, 91, 92, 93, 94, 95, 96, 97, 98, 99% homologous to at least one of these BPI3Vc isolates. More preferably, the vector will be a mosaic of a combination of these BPI3Vc isolates. One preferred BPIV3c sequence is provided as SEQ ID NO. 13 and is disclosed in PCT/US 20/70725, the teachings and content of which are hereby incorporated by reference herein.

In some forms, the BPI3Vc replicon vector includes an insert sequence from a disease-causing organism. In some forms, the organism is a coronavirus. In some forms, the coronavirus is SARS-CoV-2. In some forms, the SARS-CoV-2 insert sequence is at least one subunit of SARS-CoV-2. In some forms, the subunit is the RBD. In some forms, the RBD sequence has at least 90, 91, 92, 93, 94, 95, 96, 97, 98, 99, 99.5, 99.6, 99.7, 99.8, 99.9, or even 100% sequence homology or sequence identity with SEQ ID NO. 3.

The BPI3Vc replicon vector of the present disclosure can be administered to an animal in need thereof by any conventional method including mucosal or parenteral delivery. In some forms, the BPIV3c vector

In some forms, a plasmid DNA construct, p_{FLP}RBD_{FTM}-2A-_{CD5}SPD-CD40L, encoding the expression cassette [FIG. 1] described above is provided. This construct can be used as a plasmid DNA vaccine or it can be used to generate mRNA-based vaccine. Preliminary data shows that the encoded protein is expressed on the surface of transfected cells as judged by staining using anti-His-tag monoclonal antibody [mAb] and by anti-corona virus S protein mAbs as mentioned above [FIG. 2].

In some forms, a recombinant protein, generated using HEK Expi293F cells, transfected with a plasmid construct encoding the RBD fused in frame to the human surfactant protein D [SPD] tetramerization domain linked to human CD40L [FIG. 3], is provided. Secretion of the chimeric protein is mediated by the CD5 signal and the SPD domain is expected to result in generation of a stable soluble recombinant tetrameric RDB-SPD-CD40L chimeric protein that can be affinity purified and evaluated for its ability to induce RBD-specific neutralizing antibodies. The CD40L is expected to target the antigen to CD40 receptor expressed by professional antigen presenting cells [dendritic cells, macrophages, and B cells]. More importantly, CD40L is a powerful B cell agonist and antibody isotype switch factor (4).

In some forms, a recombinant BPI3Vc virus construct encoding the secreted tetrameric RDB-SPD-CD40L [from #2 above] [FIG. 4] is provided. The secreted tetrameric RDB-SPD-CD40L is expected to be targeted to CD40 receptor, serve as an agonist, and in addition provide isotype switching through the CD40L as mentioned in #2 above.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is an illustration of a preferred amino acid sequence of the RBD_SPD-CD40L polypeptide;

FIG. 2A is a photograph illustrating the expression and authenticity of the RBD and the CD40L was evaluated by immunocytometric analysis of HEK-293A cells transfected with the plasmid construct encoding the RBD-SPD-CD40L chimeric polypeptide using unfixed cells probed with anti-His tag monoclonal antibody (mAb) to evaluate surface display of the RBD on transfected cells;

FIG. 2B is a photograph illustrating the expression and authenticity of the RBD and the CD40L was evaluated by immunocytometric analysis of HEK-293A cells transfected with the plasmid construct encoding the RBD-SPD-CD40L chimeric polypeptide using unfixed cells probed with anti-SARS-CoV spike protein-specific polyclonal antibody (as found on the web at prosci-inc.com/sars-cov-spike-antibody-3223) to validate surface RBD expression;

FIG. 2C is a photograph illustrating the expression and authenticity of the RBD and the CD40L was evaluated by immunocytometric analysis of HEK-293A cells transfected with the plasmid construct encoding the RBD-SPD-CD40L chimeric polypeptide using fixed transfected cells probed with anti-FLAG mAb to confirm expression of the CD40L;

FIG. 2D is a photograph illustrating the expression and authenticity of the RBD and the CD40L was evaluated by immunocytometric analysis of HEK-293A cells transfected with the plasmid construct encoding the RBD-SPD-CD40L chimeric polypeptide using a negative control;

FIG. 3 is an illustration of the design of the BPI3Vc-_{FLP}RBD_{FTM}-2A-_{CD5}SPD-CD40L virus;

FIG. 4 is an illustration of a preferred amino acid sequence of the CD5-RBD_SPD-CD40L polypeptide; and

FIG. 5 is an illustration of the design of the BPI3Vc-_{CD5}RBD_SPD-CD40L polypeptide.

### DETAILED DESCRIPTION

The following detailed description and examples set forth preferred materials and procedures used in accordance with the present disclosure. It is to be understood, however, that this description and these examples are provided by way of illustration only, and nothing therein shall be deemed to be a limitation upon the overall scope of the present disclosure.

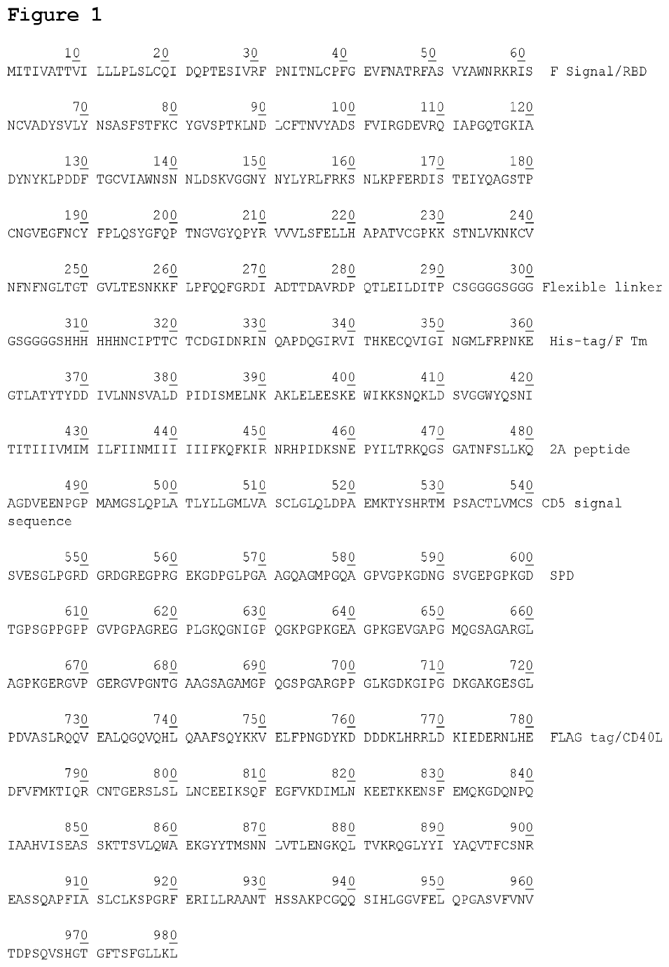

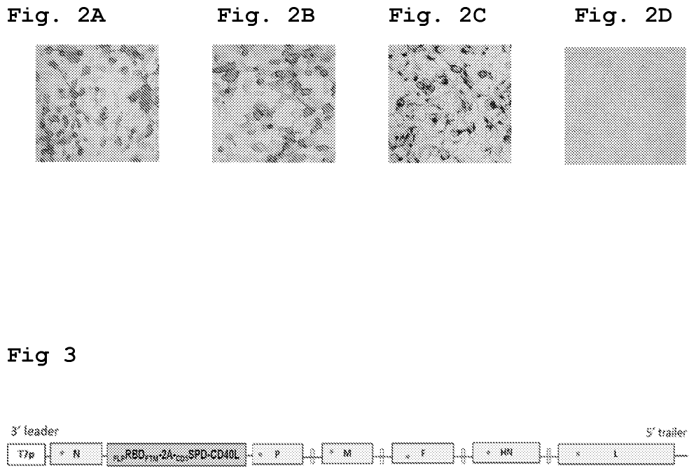

In FIG. 1, the SARS-CoV-2 RBD [S protein amino acid 319-591] (SEQ ID. NO. 3) has the BPI3Vc leader peptide at the N-terminus, a flexible linker, a His-tag, and the transmembrane domain of the BPI3Vc Fusion protein at the C terminus. A 2A autocleavable sequence was added to allow co-expression of human CD40L tetramer using human surfactant protein D [SPD] tetramerization motif. The amino acids designated as F Signal (SEQ ID NO. 2) comprise the BPI3Vc Fusion [F] protein signal sequence. The amino acids designated as RBD comprise the SARS-CoV-2 RBD [S protein amino acid 319-591] (SEQ ID NO. 3). It is understood that other coronavirus RBD sequences could be used as could other RBD sequences that have at least 90, 91, 92, 93, 94, 95, 96, 97, 98, 99, 99.5, 99.9 or even 100% sequence identity with the SEQ ID NO. 3. The amino acids designated as Flexible linker (SEQ ID NO. 4) comprise a (G₄S)₃ linker sequence to allow the RBD to fold. Although the preferred expression cassette depicted in FIG. 1 uses 3 repeating G₄S sequences, a different number of these sequences is contemplated. Additionally, it is understood that other linker sequences that are known in the art could be used. The amino acids designated as His-tag (SEQ ID NO. 5) are the amino acids comprising the Histidine tag, which allow evaluation of RBD expression. Of course, any tag will work for purposes of the present disclosure. Additionally, it is understood that the tag sequence is not critical for operation of the cassette or the effectiveness of a vaccine utilizing the cassette sequence. Accordingly, inclusion of the tag sequence is optional as it can be eliminated. The amino acids designated as F Tm (SEQ ID NO. 6) comprise the BPI3Vc Fusion [F] protein transmembrane domain for expression of the RBD on the surface of the infected cells for optimal recognition by B cells which will respond by producing anti-RBD antibodies. Other transmembrane domains could also be used, as would be understood by those of skill in the art. However, the sequence depicted below is preferred as are sequences having at least 90, 91, 92, 93, 94, 95, 96, 97, 98, 99, 99.5, 99.9 or even 100% sequence identity with the F Tm sequence depicted in FIG. 1. The amino acids designated as the 2A peptide (SEQ ID NO. 7) comprise a 2A autocleavable peptide, which allow multicistronic expression of the surface displayed RBD and secreted tetrameric CD40L molecular adjuvant. Other autocleavable peptides would also work for purposes of the disclosure. Alternatively, this sequence could be eliminated. The amino acids designated as CD5 signal sequence (SEQ ID NO. 8) comprise a CD5 secretory signal sequence. This signal sequence could be exchanged for a different signal sequence or eliminated altogether, however, this sequence is preferred. The amino acids designated as SPD (SEQ ID NO. 9) comprise a Surfactant protein D tetramerization domain. Other tetramizing domains could be utilized and be effective. The amino acids designated FLAG tag (SEQ ID NO. 10) comprise a FLAG tag to track expression of the CD40L. As noted above for the His tag, it is understood that the tag sequence is not critical for operation of the cassette or the effectiveness of a vaccine utilizing the cassette sequence. Accordingly, the tag sequence is optional and can be eliminated. The amino acids designated CD40L (SEQ ID NO. 11) comprise a molecular adjuvant/B cell agonist. It is understood that other B cell agonists could be used as could other CD40L sequences that have at least 90, 91, 92, 93, 94, 95, 96, 97, 98, 99, 99.5, 99.9 or even 100% sequence identity with the CD40L sequence depicted in FIG. 1.

In FIG. 3, the gene encoding the _{FLP}RBD_{FTM}-2A-_{CD5}SPD-CD40L expression cassette is located between N and P, which has been shown to be suitable transgene insertion site for generation of recombinant BPI3V constructs. The organization of one preferred expression cassette (SEQ ID NO. 12) is preferably as follows: FLP [F protein leader peptide]; FTM [F protein transmembrane domain]; 2A [autocleavable motif]; SPD [surfactant protein D]; and CD40L [functional domain of human CD40L]. The green dots indicate location of attenuating mutations based on the current BPI3Va vaccine virus strain [Kansas/15626/84]. Specifically, I 1103 V mutation in the polymerase gene (L) is responsible for temperature sensitive [Ts mutant] attenuation and it has been used to develop commercial BPIV3 vaccines.

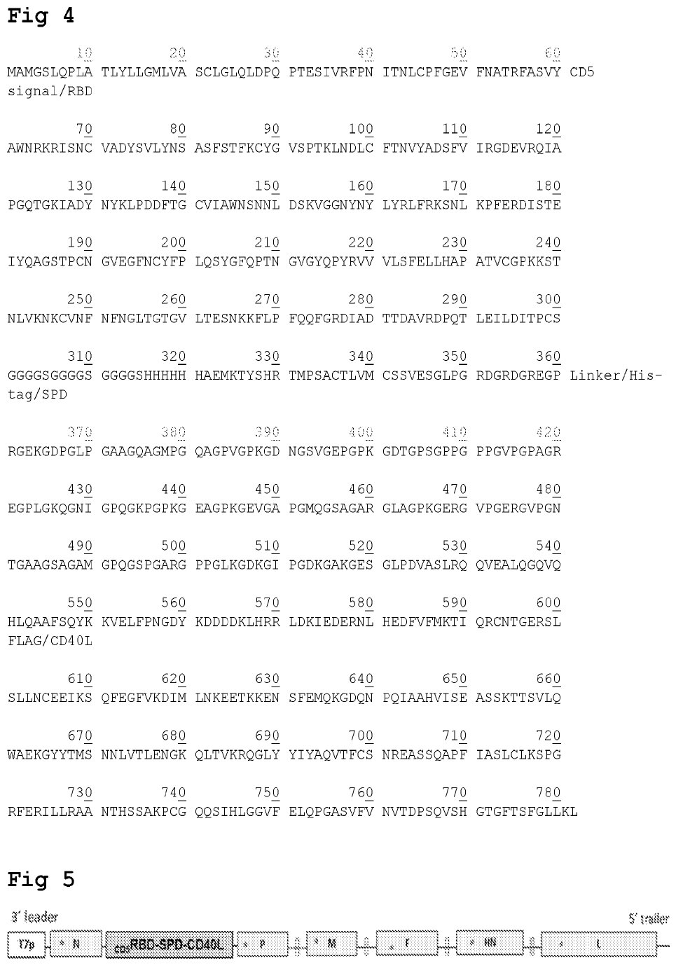

In FIG. 4, the CD5 secretory signal is at the N-terminus, followed by the SARS-CoV-2 RBD, a flexible linker, a His-tag, SPD, FLAG-tag and the CD40L functional domain.

In FIG. 5, the gene encoding the _{CD5}RBD-SPD-CD40L expression cassette is located between N and P as in FIG. 3 above. Organization of the expression cassette is as follows: CD5 [CD5 signal sequence]; SPD [surfactant protein D; and CD40L [functional domain of human CD40L].

It is understood that vaccines or immunogenic compositions produced by the present disclosure may not include all of the portions of the _{FLP}RBD_{FTM}-2A-_{CD5}SPD-CD40L provided herein. For example, the immunogenic composition may only include a signal protein, the RBD, a second signal protein and CD40L. Other forms may include a signal protein, the RBD, a second signal protein, CD40L, and at least one additional component selected from the group consisting of a flexible linker, a tag, a second tag that is different from the first tag, a transmembrane domain, an autocleavable sequence, a tetramizing domain, and any combination thereof.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

It will be found that the immunogenic compositions comprising any of the disclosed vaccine candidates as provided herewith are very effective in reducing the severity of or incidence of clinical signs associated with coronavirus infections including COVID-19 up to and including the prevention of such signs. Further, such immunogenic compositions reduce the transmissibility of COVID-19.

The immunogenic compositions described herein can further include one or more other immunomodulatory agents such as, e. g., interleukins, interferons, or other cytokines. The immunogenic compositions can also include Gentamicin and Merthiolate. In another preferred embodiment, the present disclosure contemplates vaccine compositions comprising from about 1 ug/ml to about 60 μg/ml of antibiotics, and more preferably less than about 30 μg/ml of antibiotics.

The terms “immunogenic protein”, “immunogenic polypeptide” or “immunogenic amino acid sequence” as used herein refer to any amino acid sequence which elicits an immune response in a host against a pathogen comprising said immunogenic protein, immunogenic polypeptide or immunogenic amino acid sequence. An “immunogenic protein”, “immunogenic polypeptide” or “immunogenic amino acid sequence” as used herein, includes the full-length sequence of any proteins, analogs thereof, or immunogenic fragments thereof. By “immunogenic fragment” is meant a fragment of a protein which includes one or more epitopes and thus elicits the immunological response against the relevant pathogen. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, Jun. 28-Jul. 3, 1998. It is understood that immunogenic proteins of the present disclosure include the RBD sequence, the F-Tm sequence, the SPD sequence, and the CD40L sequence.

In the present description, the terms polypeptide, peptide and protein are interchangeable.

In the present description, COVID-19 (the disease) and SARS-CoV2 (the causative agent) are used interchangeably.

Additionally, any composition or vaccine candidate of the disclosure can include one or more pharmaceutical-acceptable or veterinary-acceptable carriers. As used herein, “a pharmaceutical-acceptable carrier” or “veterinary-acceptable carrier” includes any and all solvents, dispersion media, coatings, stabilizing agents, diluents, preservatives, antibacterial and antifungal agents, isotonic agents, adsorption delaying agents, and the like. In some forms, the pharmaceutical or veterinary acceptable carrier is selected from the group consisting of a solvent, a dispersion media, a coating, a stabilizing agent, a preservative, an antimicrobial agent, an antifungal agent, an isotonic agent, and an adsorption delaying agent, and any combination thereof.

It is understood that the immunogenic compositions described herein can be administered to any animal susceptible to coronavirus infection including humans, dogs, cats, ferrets, bats, cattle, camels, hamsters, horses, chimps, gorillas, anteaters, dolphins, alligators, and sheep. Further, administration of any of the immunogenic compositions described herein will reduce the incidence of, severity of, and transmission of SARS-CoV2.

Immunogenic Compositions and Methods of Making and Using Such Compositions

One aspect of the present disclosure provides a method of producing and/or recovering recombinant SARS-CoV-2 RBD protein, by 1) infecting a number of susceptible cells in culture with a recombinant viral vector encoding a SARS-CoV-2 RBD protein, 2) expressing SARS-CoV-2 RBD protein by the recombinant viral vector, 3) recovering the SARS-CoV-2 RBD protein, and, 4) separating cell debris from the expressed SARS-CoV-2 RBD protein via a separation step.

In some forms, the recombinant viral vector is the BPIV3c vector. In preferred forms, the vector has the sequence of SEQ ID NO. 13. In some preferred forms, the vector has at least 90, 91, 92, 93, 94, 95, 96, 97, 98, 99, 99.5, 99.6, 99.7, 99.8, 99.9 or even 100% sequence identity or homology with SEQ ID NO. 13.

In another aspect of the present disclosure, the inclusion of an inactivation step is preferred in order to inactivate the viral vector prior to recovery of SARS-CoV-2 RBD protein that will be used in an immunogenic or immunological composition such as a vaccine. Such a step can be performed as step 5) in addition to steps 1-4 described above.

In some forms, this inactivation is done either just before or just after the filtration or separation step. Any conventional inactivation method can be used for purposes of the present disclosure. Thus, inactivation can be performed by chemical and/or physical treatments. One representative inactivation method includes the addition of cyclized binary ethylenimine (BEI).

Optionally, the method described above may also include a neutralization step after step 5). For example, if the inactivation agent is BEI, addition of sodium thiosulfate to an equivalent amount is preferred. Preferably, the sodium thiosulfate is added in equivalent amount as compared to the BEI added for inactivation.

An “immunogenic or immunological composition” refers to a composition of matter that comprises at least one antigen which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or γδ T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the severity or prevalence of, up to and including a lack of symptoms normally displayed by an infected host, a quicker recovery time and/or a lowered viral titer in the infected host.

A “reduction” in terms of incidence of symptoms, severity of symptoms, or transmissibility of infection is understood to encompass a comparison to a subject or group of subjects that has not received an administration of a composition of the present disclosure. Preferably, the reduction is at least 10, 20, 30, 40, 50, 60, 70, 80, 90, 95, or even 100% in comparison to an animal that has not received an administration or dose of a composition described herein. It is understood that this percentage of reduction can be in terms of the number of symptoms, incidence of symptoms, severity of symptoms, and transmissibility of infection. Further, this can apply to individual animals or groups of animals.

In preferred forms and especially in forms that will use the recombinant SARS-CoV-2 RBD protein in an immunogenic composition such as a vaccine, each lot or just selected lots of harvested SARS-CoV-2 RBD protein can be tested for inactivation. Thus, a further aspect of the present disclosure relates to an inactivation test for determining the effectiveness of the inactivation of the recombination viral vector, comprising the steps: 1) contacting at least a portion of the culture fluid containing the recombinant viral vector with an inactivating agent, 2) adding a neutralization agent to neutralize the inactivation agent, and 3) determining the residual infectivity.

In preferred forms the recombinant viral vector containing SARS-CoV-2 RBD DNA and expressing SARS-CoV-2 RBD protein used to infect the cells is generated by transfecting a transfer vector that has had a SARS-CoV-2 RBD gene cloned therein into a viral vector. In some preferred forms, only the portion of the transfer vector that contains the desired SARS-CoV-2 RBD DNA is transfected into the viral vector.

The term “transfected into a viral vector” means, and is used as a synonym for “introducing” or “cloning” a heterologous DNA into a viral vector, such as for example into a baculovirus vector. A “transfer vector” means a DNA molecule, that includes at least one origin of replication, the heterologous gene, in the present case of SARS-CoV-2 RBD, DNA sequences which allow the cloning of said heterologous gene into the viral vector will be included. Preferably the sequences which allow cloning of the heterologous gene into the viral vector are flanking the heterologous gene. Even more preferably, those flanking sequences are at least homologous in parts with sequences of the viral vector. The sequence homology then allows recombination of both molecules, the viral vector, and the transfer vector to generate a recombinant viral vector containing the heterologous gene.

In more preferred forms, the methods of the present disclosure will begin with the isolation of SARS-CoV-2 RBD DNA. Any SARS-CoV-2 RBD gene, such as variants of the SARS-CoV-2 that causes COVID-19, can be used for purposes of the present disclosure. The SARS-CoV-2 RBD DNA is preferably amplified using PCR methods. The resulting DNA is then cloned into the transfer vector.

Thus, in one aspect of the present disclosure, a method for constructing a recombinant viral vector containing SARS-CoV-2 RBD DNA is provided. This method generally comprises the steps of: 1) cloning at least one recombinant SARS-CoV-2 RBD gene into a transfer vector; and 2) transfecting the portion of the transfer vector containing the recombinant SARS-CoV-2 RBD gene into a viral vector, to generate the recombinant viral vector.

According to a further aspect, the SARS-CoV-2 RBD DNA can be amplified prior to step 1) in vitro, wherein the flanking sequences of the SARS-CoV-2 RBD DNA are modified. In vitro methods for amplifying the SARS-CoV-2 RBD DNA and modifying the flanking sequences, cloning in vitro amplified SARS-CoV-2 RBD DNA into a transfer vector and suitable transfer vectors are described above or known to a person skilled in the art. Thus according to a further aspect, the present disclosure relates to a method for constructing a recombinant viral vector containing SARS-CoV-2 RBD DNA and expressing a desired SARS-CoV-2 RBD protein comprising the steps of: 1) amplifying SARS-CoV-2 RBD DNA in vitro, wherein the flanking sequences of said SARS-CoV-2 RBD DNA are modified, 2) cloning the amplified SARS-CoV-2 RBD DNA into a transfer vector; and 3) transfecting the transfer vector or a portion thereof containing the recombinant SARS-CoV-2 RBD DNA into a viral vector to generate the recombinant viral vector. In some aspects, the modification of the flanking sequences of the SARS-CoV-2 RBD DNA is performed by introducing a 5′ Kozak's sequence and/or an EcoR 1 site.

A further aspect of the present disclosure relates to a method for preparing a composition comprising SARS-CoV-2 RBD protein, and inactivated viral vector. This method generally comprises the steps of: 1) cloning the amplified SARS-CoV-2 RBD DNA into a transfer vector; 2) transfecting the portion of the transfer vector containing the recombinant SARS-CoV-2 RBD DNA into a virus; 3) infecting cells in media with the transfected viral vector; 4) causing the transfected viral vector to express the recombinant protein from the SARS-CoV-2 RBD DNA; 5) separating cells from the supernate; 6) recovering the expressed SARS-CoV-2 RBD protein; and 7) inactivating the recombinant viral vector. In preferred forms and as described above, a neutralization step, step 8), will be performed after step 7). Of course, prior to step 1) the SARS-CoV-2 RBD DNA can be amplified in vitro, preferably with flanking sequences of the SARS-CoV-2 RBD DNA, as described above.

In another aspect of the present disclosure, a method for preparing a composition, preferably an immunogenic composition, such as a vaccine, for invoking an immune response against SARS-CoV-2 is provided. Generally, this method includes the steps of transfecting a construct into a virus, wherein the construct comprises 1) recombinant DNA from an RBD of SARS-CoV-2, 2) infecting cells in growth media with the transfected virus, 3) causing the virus to express the recombinant RBD protein from SARS-CoV-2, 4) recovering the expressed recombinant RBD protein, 5) and preparing the composition by combining the recovered protein with a suitable adjuvant and/or other pharmaceutically acceptable carrier. In some preferred forms, the composition also includes at least a portion of the viral vector expressing said SARS-CoV-2 RBD protein, and/or a portion of the cell culture supernate

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). John Wiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose. In another aspect of the present disclosure, a method for preparing an immunogenic composition, such as a vaccine, for invoking an immune response against SARS-CoV-2 comprises the steps of 1) expressing and recovering SARS-CoV-2 RBD protein, and in preferred forms, 2) admixing the recovered protein with a suitable adjuvant. Preferably, the expressing step 1) includes steps for the preparation and recovery of SARS-CoV-2 RBD protein. Another optional step for this method includes cloning the amplified SARS-CoV-2 RBD DNA into a first vector, excising the RBD DNA from this first vector, and using this excised SARS-CoV-2 RBD DNA for cloning into the transfer vector. Preferably, the recovery step of this method also includes the step of separating the media from the cells and cell debris. This can be done in any conventional manner, with one preferred manner comprising filtering the cells, cell debris, and growth media through a filter having pores ranging in size from about 0.45 μM to about 1.0 μM. Finally, for this aspect, it is preferred to include a virus inactivation step prior to combining the recovered recombinant SARS-CoV-2 RBD protein in a composition. When an inactivation step is included, it is also preferred to include a neutralization step, as described above.

Additionally, the composition can include one or more pharmaceutical-acceptable or veterinary-acceptable carriers. As used herein, “a pharmaceutical-acceptable carrier” or “veterinary-acceptable carrier” includes any and all solvents, dispersion media, coatings, stabilizing agents, diluents, preservatives, antibacterial and antifungal agents, isotonic agents, adsorption delaying agents, and the like. In one preferred form, the composition provided herewith, contains SARS-CoV-2 RBD protein recovered from in vitro cultured cells, wherein said cells were infected with a recombinant viral vector containing SARS-CoV-2 RBD DNA and expressing SARS-CoV-2 RBD protein, and wherein the cell culture was treated to inactivate the viral vector, and an equivalent concentration of a neutralization agent was added, and wherein both an adjuvant and physiological saline are also added. As with the other aspects, the SARS-CoV-2 RBD protein can have at least 90, 91, 92, 93, 94, 95, 96, 97, 98, 99, 99.5, 99.6, 99.7, 99.8, 99.9, or even 100% sequence identity or sequence homology with SEQ ID NO. 3. When included, the amount of physiological saline is preferably about 50 to about 90% (v/v), more preferably about 60 to 80% (v/v), still more preferably about 70% (v/v). Optionally, this method can also include the addition of a protectant. A protectant as used herein, refers to an anti-microbiological active agent, such as for example Gentamycin, Merthiolate, and the like. In particular adding a protectant is most preferred for the preparation of a multi-dose composition. Those anti-microbiological active agents are added in concentrations effective to prevent the composition of interest from any microbiological contamination or for inhibition of any microbiological growth within the composition of interest.

The methods of the present disclosure can also comprise the addition of any stabilizing agent, such as for example saccharides, trehalose, mannitol, saccharose and the like, to increase and/or maintain product shelf-life and/or to enhance stability.

In another aspect of the present disclosure, products resulting from the methods as described above are provided. In particular, the present disclosure relates to a composition of matter comprising recombinantly expressed SARS-CoV-2 RBD protein. In some preferred forms, this composition of matter also comprises an agent suitable for the inactivation of viral vectors and comprises an agent, suitable for the inactivation of viral vectors. Such products are useful as immunogenic compositions that induce an immune response and, more preferably, confers protective immunity against the clinical signs of SARS-CoV-2 infection. The composition generally comprises the polypeptide, or a fragment thereof, expressed by the RBD gene of SARS-CoV-2, as the antigenic component of the composition. Of course, it is understood that the SARS-CoV-2 RBD polypeptide used in an immunogenic composition in accordance with the present disclosure can be derived in any fashion including isolation and purification, standard protein synthesis, and recombinant methodology.

Any SARS-CoV-2 RBD would be effective as the source of the SARS-CoV-2 RBD DNA and/or polypeptide as used herein. In preferred forms, the RBD DNA encodes a polypeptide having at least 90, 91, 92, 93, 94, 95, 96, 97, 98, 99, 99.5, 99.6, 99.7, 99.8, 99.9, or even 100% sequence homology or sequence identity with SEQ ID NO. 3. A preferred SARS-CoV-2 RBD protein is that of SEQ ID NO. 3, but it is understood by those of skill in the art that this sequence could vary by as much as 10% in sequence homology and still retain the antigenic characteristics that render it useful in immunogenic compositions. The antigenic characteristics of an immunological composition can be, for example, estimated by challenge experiments. Moreover, the antigenic characteristic of a modified antigen is still retained, when the modified antigen confers at least 70%, preferably 80%, more preferably 90% of the protective immunity as compared to the SARS-CoV-2 RBD protein of SEQ ID NO. 3. In some forms, immunogenic portions of a SARS-CoV-2 RBD protein are used as the antigenic component in the composition. The term “immunogenic portion” as used herein refers to truncated and/or substituted forms, or fragments of SARS-CoV-2 RBD protein and/or polynucleotide, respectively. Preferably, such truncated and/or substituted forms, or fragments will comprise at least 6 contiguous amino acids from the full-length RBD polypeptide. More preferably, the truncated or substituted forms, or fragments will have at least 10, more preferably at least 15, and still more preferably at least 19 contiguous amino acids from the full-length RBD polypeptide. It is further understood that such sequences may be a part of larger fragments or truncated forms. Preferably, such truncated or substituted forms, or fragments will comprise at least 18 contiguous nucleotides from the full-length RBD nucleotide sequence, e.g. of SEQ ID NO. 3. More preferably, the truncated or substituted forms, or fragments will have at least 30, more preferably at least 45, and still more preferably at least 57 contiguous nucleotides the full-length RBD nucleotide sequence.

In a further aspect of the present disclosure, an immunogenic composition effective for lessening the severity and/or reducing the incidence of clinical symptoms associated with SARS-CoV-2 infection, and/or reducing the transmissibility of SARS-CoV-2, comprising SARS-CoV-2 RBD protein is provided. Preferably, the SARS-CoV-2 RBD protein is selected from the group consisting of: 1) a polypeptide comprising the sequence of SEQ ID NO: 3; 2) any polypeptide that is at least 90% homologous to the polypeptide of 1); 3) any immunogenic portion of the polypeptides of 1) and/or 2); or 4) the immunogenic portion of 3), comprising at least 10 contiguous amino acids included in the sequence of SEQ ID NO: 3.

In preferred forms, these immunogenic portions will have the immunogenic characteristics of SARS-CoV-2 RBD protein that has at least 90, 91, 92, 93, 94, 95, 96, 97, 98, 99, 99.5, 99.6, 99.7, 99.8, 99.9, or even 100% sequence homology with SEQ ID NO. 3.

According to a further aspect, SARS-CoV-2 RBD protein is provided in the immunological composition at an antigen inclusion level effective for inducing the desired immune response, namely reducing the incidence of or lessening the severity of clinical signs resulting from SARS-CoV-2 infection, or reducing the transmissibility of SARS-CoV-2. In some preferred forms, the SARS-CoV-2 RBD protein inclusion level is at least 0.2 μg antigen/ml of the final immunogenic composition (μg/ml), more preferably from about 0.2 to about 2000 μg/ml.

The polypeptide is incorporated into a composition that can be administered to an animal susceptible to SARS-CoV-2 infection. In preferred forms, the composition may also include additional components known to those of skill in the art (see also Remington's Pharmaceutical Sciences. (1990). 18th ed. Mack Publ., Easton).

Those of skill in the art will understand that the composition herein may incorporate known injectable, physiologically acceptable, sterile solutions. For preparing a ready-to-use solution for parenteral injection or infusion, aqueous isotonic solutions, such as e.g. saline or corresponding plasma protein solutions are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others. Suitable adjuvants, are those described above.

According to a further aspect, the immunogenic composition of the present disclosure further comprises a pharmaceutical acceptable salt, preferably a phosphate salt in physiologically acceptable concentrations. Preferably, the pH of said immunogenic composition is adjusted to a physiological pH, meaning between about 6.5 and 7.5.

The immunogenic compositions described herein can further include one or more other immunomodulatory agents such as, e. g., interleukins, interferons, or other cytokines. The immunogenic compositions can also include Gentamicin and Merthiolate. In another preferred embodiment, the present disclosure contemplates vaccine compositions comprising from about 1 ug/ml to about 60 μg/ml of antibiotics, and more preferably less than about 30 μg/ml of antibiotics.

It will be found that the immunogenic compositions comprising recombinant SARS-CoV-2 RBD protein as provided herewith are very effective in reducing the severity of or incidence of clinical signs associated with SARS-CoV-2 infections up to and including the prevention of such signs. Additionally, the immunogenic compositions will be effective at reducing the transmissibility of SARS-CoV-2.

Another aspect of the present disclosure relates to a kit. Generally, the kit includes a container comprising at least one dose of the immunogenic composition of SARS-CoV-2 RBD protein as provided herewith, wherein one dose comprises at least 2 μg SARS-CoV-2 RBD protein. Said container can comprise from 1 to 250 doses of the immunogenic composition. In some preferred forms, the container contains 1, 10, 25, 50, 100, 150, 200, or 250 doses of the immunogenic composition of SARS-CoV-2 RBD protein. Preferably, each of the containers comprising more than one dose of the immunogenic composition of SARS-CoV-2 RBD protein further comprises an anti-microbiological active agent. Those agents are for example, antibiotics including Gentamicin and Merthiolate and the like. Thus, one aspect of the present disclosure relates to a container that comprises from 1 to 250 doses of the immunogenic composition of SARS-CoV-2 RBD protein, wherein one dose comprises at least 2 μg SARS-CoV-2 RBD protein, and Gentamicin and/or Merthiolate, preferably from about 1 μg/ml to about 60 μg/ml of antibiotics, and more preferably less than about 30 μg/ml. In preferred forms, the kit also includes an instruction manual, including the information for the intramuscular application of at least one dose of the immunogenic composition of SARS-CoV-2 RBD protein into animals, to lessen the incidence and/or severity of clinical symptoms associated with SARS-CoV-2 infection. Moreover, according to a further aspect, said instruction manual comprises the information of a second or further administration(s) of at least one dose of the immunogenic composition of SARS-CoV-2 RBD, wherein the second administration or any further administration is at least 14 days beyond the initial or any former administration. In some preferred forms, said instruction manual also includes the information, to administer an immune stimulant. Preferably, said immune stimulant shall be given at least twice. Preferably, at least 3, more preferably at least 5, and even more preferably at least 7 days are between the first and the second or any further administration of the immune stimulant. Preferably, the immune stimulant is given at least 10 days, preferably 15, even more preferably 20, and still even more preferably at least 22 days beyond the initial administration of the immunogenic composition of SARS-CoV-2 RBD protein. It is understood that any immune stimulant known to a person skilled in the art can also be used. “Immune stimulant” as used herein, means any agent or composition that can trigger a general immune response, preferably without initiating or increasing a specific immune response, for example the immune response against a specific pathogen. It is further instructed to administer the immune stimulant in a suitable dose. The kit may also comprise a second container, including at least one dose of the immune stimulant.

A further aspect of the present disclosure relates to the kit as described above, comprising the immunogenic composition of SARS-CoV-2 RBD as provided herewith and the instruction manual, wherein the instruction manual further includes the information to administer the SARS-CoV-2 RBD immunogenic composition together, or around the same time as, with an immunogenic composition that comprises an additional antigen effective for reducing the severity of or incidence of clinical signs related to another mammalian pathogen. Preferably, the manual contains the information of when the SARS-CoV-2 RBD containing composition and the immunogenic composition that comprises an additional antigen are administered.

A further aspect, relates to the use of any of the compositions provided herewith as a medicament, preferably as a veterinary medicament, even more preferably as a vaccine. Moreover, the present disclosure also relates to the use of any of the compositions described herein, for the preparation of a medicament for lessening the severity of clinical symptoms associated with SARS-CoV-2 infection. Preferably, the medicament is for the prevention of a SARS-CoV-2 infection in an animal susceptible to infection with SARS-CoV-2.

A further aspect relates to a method for (1) the prevention of an infection, or re infection with SARS-CoV-2 or (2) the reduction in incidence or severity of or elimination of clinical symptoms caused by SARS-CoV-2 in a subject, comprising administering any of the immunogenic compositions provided herewith to a subject in need thereof. It is understood that the reduction is in comparison to a subject that has not received an administration of a composition of the present disclosure. Preferably, the reduction is at least 10, 20, 30, 40, 50, 60, 70, 80, 90, 95, or even 100% in comparison to an animal that has not received an administration or dose of a composition described herein. It is understood that this percentage of reduction can be in terms of the number of symptoms, incidence of symptoms, or severity of symptoms. Further, this can apply to individual animals or groups of animals. Preferably, one dose or two doses of the immunogenic composition is/are administered, wherein one dose preferably comprises at least about 2 μg SARS-CoV-2 RBD protein. A further aspect relates to the method of treatment as described above, wherein a second application of the immunogenic composition is administered. Preferably, the second administration is done with the same immunogenic composition, preferably having the same amount of SARS-CoV-2 RBD protein. Preferably, the second administration is done at least 14 days beyond the initial administration, even more preferably at least 4 weeks beyond the initial administration. In preferred forms, the method is effective after just a single dose of the immunogenic composition and does not require a second or subsequent administration in order to confer the protective benefits upon the subject.

According to a further aspect, the present disclosure provides a multivalent combination vaccine which includes an immunological agent effective for reducing the incidence of or lessening the severity of SARS-CoV-2 infection, and at least one immunological active component against another disease-causing organism in mammals. In some forms, the DNA encoding SARS-CoV-2 RBD protein and the at least one immunological active component are integrated into, or transfected into the BPIV3C vector for administration thereof or for expressing the desired immunogenic components of SARS-CoV-2 and the at least one immunological active component.

In particular the immunological agent effective for reducing the incidence of or lessening the severity of SARS-CoV-2 infection is a SARS-CoV-2 antigen. Preferably, said SARS-CoV-2 antigen is a SARS-CoV-2 RBD protein as provided herewith, or any immunogenic composition as described above, that comprises SARS-CoV-2 RBD protein, such as a SARS-CoV-2 RBD protein that has at least 90, 91, 92, 93, 94, 95, 96, 97, 98, 99, 99.5, 99.6, 99.7, 99.8, 99.9, or even 100% sequence homology with SEQ ID NO. 3.

An “immunological active component” as used herein means a component that induces or stimulates the immune response in an animal to which said component is administered. According to a preferred embodiment, said immune response is directed to said component or to a microorganism comprising said component. According to a further preferred embodiment, the immunological active component is an attenuated microorganism, including modified live virus (MLV), a killed-microorganism or at least an immunological active part of a microorganism.

“Immunological active part of a microorganism” as used herein means a protein-, sugar-, and or glycoprotein containing fraction of a microorganism that comprises at least one antigen that induces or stimulates the immune response in an animal to which said component is administered. According to a preferred embodiment, said immune response is directed to said immunological active part of a microorganism or to a microorganism comprising said immunological active part.

Also included within the scope of the present disclosure are biologically functional plasmids, viral vectors and the like that contain the new recombinant nucleic acid molecules described herein, suitable host cells transfected by the vectors comprising the molecular DNA clones and the immunogenic polypeptide expression products. Some particularly preferred immunogenic proteins will have the amino acid sequence set forth in SEQ ID NO: 3. The biologically active variants thereof are further encompassed by the disclosure. One of ordinary skill in the art would know how to modify, substitute, delete, etc., amino acid(s) from the polypeptide sequence and produce biologically active variants that retain the same, or substantially the same, activity as the parent sequence without undue effort.

To produce the immunogenic polypeptide products of this disclosure, the process may include the following steps: growing, under suitable nutrient conditions, prokaryotic or eukaryotic host cells transfected with the new recombinant nucleic acid molecules described herein in a manner allowing expression of said polypeptide products, and isolating the desired polypeptide products of the expression of said nucleic acid molecules by standard methods known in the art. It is contemplated that the immunogenic proteins may be prepared by other techniques such as, for example, biochemical synthesis and the like.

The present disclosure also relates to vaccines comprising a nucleotide sequence of the genome of SARS-CoV-2 that encodes the RBD of SARS-CoV-2. In some preferred forms, the RBD protein of SARS-CoV-2 has at least 90, 91, 92, 93, 94, 95, 96, 97, 98, 99, 99.5, 99.6, 99.7, 99.8, 99.9, or even 100% sequence homology with SEQ ID NO. 3 or a homologue or fragment thereof, and an acceptable pharmaceutical or veterinary vehicle. In one embodiment of the disclosure, the nucleotide sequence is selected from, or a homologue or fragment thereof. In yet another embodiment, the vaccine comprising the nucleotide sequence encoding the SARS-CoV-2 RBD protein further comprises an adjuvant.

A further aspect of the present disclosure relates to vaccines comprising a vector and an acceptable pharmaceutical or veterinary vehicle, the vector comprising a nucleotide sequence of the genome of SARS-CoV-2, preferably the portion encoding the SARS-CoV-2 RBD protein, such as SEQ ID NO. 3. In some forms, the nucleotide sequence encodes SARS-CoV-2 RBD protein that has at least 90, 91, 92, 93, 94, 95, 96, 97, 98, 99, 99.5, 99.6, 99.7, 99.8, 99.9, or even 100% sequence homology with SEQ ID NO. 1 or 12 as well as at least one of a signal sequence, receptor binding domains, a linker, a tag, a fusion protein transmembrane, a 2A cleavable peptide, a signal sequence, a surfactant protein tetramerization domain, a FLAG tag, and CD40L. In some forms, the signal sequence is a F signal protein, preferably SEQ ID No. 2. In some forms, the linker is SEQ ID NO. 4. In some forms, the tag is a His tag, preferably SEQ ID NO. 5. In some forms, the fusion protein transmembrane is SEQ ID NO. 6. In some forms, the 2A cleavable peptide is SEQ ID NO. 7. In some forms, the signal sequence is a CD5 signal sequence, preferably SEQ ID NO. 8. In some forms, the surfactant protein tetramerization domain is SEQ ID NO. 9. In some forms, the FLAG tag is SEQ ID NO. 10. In some forms, the CD40L is SEQ ID NO. 11.

The present disclosure also relates to vaccines immunogenic compositions comprising a cell and an acceptable pharmaceutical or veterinary carrier, wherein the cell is transformed with a nucleotide sequence of the genome of SARS-CoV-2, or a homologue or fragment thereof, preferably of the RBD of SARS-CoV-2.

Still further, the present disclosure relates to vaccines or immunogenic compositions comprising a pharmaceutically acceptable vehicle and a single polypeptide, wherein the single polypeptide consists of SEQ ID No. 3.

Additionally, the present disclosure relates to methods of immunizing a mammal against SARS-CoV-2 comprising administering to a mammal an effective amount of a vaccine or immunogenic composition described above.

The present disclosure likewise relates to nucleotide sequences, characterized in that they are selected from: a) a nucleotide sequence encoding a specific fragment of the sequence of SEQ ID No. 1, 3, or 12 or one of their fragments; b) a nucleotide sequence homologous to a nucleotide sequence such as defined in a); c) a nucleotide sequence complementary to a nucleotide sequence such as defined in a) or b), and a nucleotide sequence of their corresponding RNA; d) a nucleotide sequence capable of hybridizing under stringent conditions with a sequence such as defined in a), b) or c); e) a nucleotide sequence comprising a sequence such as defined in a), b), c) or d); and f) a nucleotide sequence modified by a nucleotide sequence such as defined in a), b), c), d) or e).

Nucleotide, polynucleotide or nucleic acid sequence will be understood according to the present disclosure as meaning both a double-stranded or single-stranded DNA in the monomeric and dimeric (so-called in tandem) forms and the transcription products of said DNAs.

It must be understood that the present disclosure does not relate to the genomic nucleotide sequences taken in their natural environment, that is to say in the natural state. It concerns sequences which it has been possible to isolate, purify or partially purify, starting from separation methods such as, for example, ion-exchange chromatography, by exclusion based on molecular size, or by affinity, or alternatively fractionation techniques based on solubility in different solvents, or starting from methods of genetic engineering such as amplification, cloning and subcloning, it being possible for the sequences of the disclosure to be carried by vectors. All genes and vectors in the present application were generated using synthetic biology.

Complementary nucleotide sequence of a sequence of the disclosure is understood as meaning any DNA whose nucleotides are complementary to those of the sequence of the disclosure, and whose orientation is reversed (antiparallel sequence).

Hybridization under conditions of stringency with a nucleotide sequence according to the disclosure is understood as meaning a hybridization under conditions of temperature and ionic strength chosen in such a way that they allow the maintenance of the hybridization between two fragments of complementary DNA.

Among the nucleotide sequences according to the disclosure, those are likewise preferred which can be used as a primer or probe in methods allowing the homologous sequences according to the disclosure to be obtained, these methods, such as the polymerase chain reaction (PCR), nucleic acid cloning and sequencing, being well known to the person skilled in the art.

Among said nucleotide sequences according to the disclosure, those are again preferred which can be used as a primer or probe in methods allowing the presence of SARS-CoV-2 or one of its variants such as defined below to be diagnosed.

The nucleotide sequences according to the disclosure capable of modulating, of inhibiting or of inducing the expression of SARS-CoV-2 gene, and/or capable of modulating the replication cycle of SARS-CoV-2 in the host cell and/or organism are likewise preferred. Replication cycle will be understood as designating the invasion and the multiplication of SARS-CoV-2, and its propagation from host cell to host cell in the host organism.

Among said nucleotide sequences according to the disclosure, those corresponding to RBD sequences, and coding for polypeptides, such as, for example, SEQ ID No. 3. The nucleotide sequence fragments according to the disclosure can be obtained, for example, by specific amplification, such as PCR, or after digestion with appropriate restriction enzymes of nucleotide sequences according to the disclosure, these methods in particular being described in the work of Sambrook et al., 1989. Said representative fragments can likewise be obtained by chemical synthesis when their size is not very large and according to methods well known to persons skilled in the art.

Modified nucleotide sequence will be understood as meaning any nucleotide sequence obtained by mutagenesis according to techniques well known to the person skilled in the art, and containing modifications with respect to the normal sequences according to the disclosure, for example mutations in the regulatory and/or promoter sequences of polypeptide expression, especially leading to a modification of the rate of expression of said polypeptide or to a modulation of the replicative cycle.

Modified nucleotide sequence will likewise be understood as meaning any nucleotide sequence coding for a modified polypeptide such as defined below.

The present disclosure relates to nucleotide sequences of SARS-CoV-2 according to the disclosure, characterized in that they are selected from the sequences encoding SEQ ID No. 3 or sequences having at least 90, 91, 92, 93, 94, 95, 96, 97, 98, 99, 99.5, 99.6, 99.7, 99.8, 99.9, or even 100% sequence identity or homology to SEQ ID NO. 3, or one of their fragments.

The disclosure likewise relates to nucleotide sequences characterized in that they comprise a nucleotide sequence selected from: a) a nucleotide sequence encoding SEQ ID No. 3, or one of their fragments; b) a nucleotide sequence of a specific fragment of a sequence such as defined in a); c) a homologous nucleotide sequence having at least 80% identity with a sequence such as defined in a) or b); d) a complementary nucleotide sequence or sequence of RNA corresponding to a sequence such as defined in a), b) or c); and e) a nucleotide sequence modified by a sequence such as defined in a), b), c) or d).

As far as homology with the nucleotide sequences encoding SEQ ID No. 3 or one of their fragments is concerned, the homologous, especially specific, sequences having a percentage identity with SEQ ID No. 3, or one of their fragments of at least 90, 91, 92, 93, 94, 95, 96, 97, 98, 99, 99.5, 99.6, 99.7, 99.8, 99.9, or even 100% are preferred. Said specific homologous sequences can comprise, for example, the sequences corresponding to RBD sequences of SARS-CoV-2. In the same manner, these specific homologous sequences can correspond to variations linked to mutations within strains of SARS-CoV-2 and especially correspond to truncations, substitutions, deletions and/or additions of at least one nucleotide.

The disclosure comprises the polypeptides encoded by a nucleotide sequence according to the disclosure, preferably a polypeptide whose sequence is represented by a fragment, especially a specific fragment, these six amino acid sequences corresponding to the polypeptides which can be encoded according to SEQ ID No. 3.

The disclosure likewise relates to the polypeptides, characterized in that they comprise a polypeptide selected from the amino acid sequences SEQ ID No. 3 or one of their fragments.

The disclosure also relates to the polypeptides, characterized in that they comprise a polypeptide selected from: a) a specific fragment of at least 5 amino acids of a polypeptide of an amino acid sequence according to the disclosure; b) a polypeptide homologous to a polypeptide such as defined in a); c) a specific biologically active fragment of a polypeptide such as defined in a) or b); and d) a polypeptide modified by a polypeptide such as defined in a), b) or c).

Among the polypeptides according to the disclosure, the polypeptides of amino acid sequences SEQ ID No. 1, 3, and SEQ ID No. 12 are also preferred, these polypeptides being especially capable of specifically recognizing the antibodies produced during infection by SARS-CoV-2. These polypeptides thus have epitopes specific for the SARS-CoV-2 and can thus be used in particular in the diagnostic field or as immunogenic agent to confer protection in an animal against infection by SARS-CoV-2.

In the present description, the terms polypeptide, peptide and protein are interchangeable.

It must be understood that the disclosure does not relate to the polypeptides in natural form, that is to say that they are not taken in their natural environment but that they can be isolated or obtained by purification from natural sources, or else obtained by genetic recombination, or alternatively by chemical synthesis and that they can thus contain unnatural amino acids, as will be described below.

Polypeptide fragment according to the disclosure is understood as designating a polypeptide containing at least 5 consecutive amino acids, preferably 10 consecutive amino acids or 15 consecutive amino acids.

In the present disclosure, specific polypeptide fragment is understood as designating the consecutive polypeptide fragment encoded by a specific fragment nucleotide sequence according to the disclosure.

Homologous polypeptide will be understood as designating the polypeptides having, with respect to the natural polypeptide, certain modifications such as, in particular, a deletion, addition or substitution of at least one amino acid, a truncation, a prolongation, a chimeric fusion, and/or a mutation. Among the homologous polypeptides, those are preferred whose amino acid sequence has at least 90, 91, 92, 93, 94, 95, 96, 97, 98, 99, 99.5, 99.6, 99.7, 99.8, 99.9, or even 100%, homology with the sequences of amino acids of polypeptides according to the disclosure.

Specific homologous polypeptide will be understood as designating the homologous polypeptides such as defined above and having a specific fragment of polypeptide according to the disclosure.

In the case of a substitution, one or more consecutive or nonconsecutive amino acids are replaced by “equivalent” amino acids. The expression “equivalent” amino acid is directed here at designating any amino acid capable of being substituted by one of the amino acids of the base structure without, however, essentially modifying the biological activities of the corresponding peptides and such that they will be defined by the following. These equivalent amino acids can be determined either by depending on their structural homology with the amino acids which they substitute, or on results of comparative tests of biological activity between the different polypeptides, which are capable of being carried out. By way of example, the possibilities of substitutions capable of being carried out without resulting in an extensive modification of the biological activity of the corresponding modified polypeptides will be mentioned, the replacement, for example, of leucine by valine or isoleucine, of aspartic acid by glutamic acid, of glutamine by asparagine, of arginine by lysine etc., the reverse substitutions naturally being envisageable under the same conditions.

The specific homologous polypeptides likewise correspond to polypeptides encoded by the specific homologous nucleotide sequences such as defined above and thus comprise in the present definition the polypeptides which are mutated or correspond to variants which can exist in SARS-CoV-2, and which especially correspond to truncations, substitutions, deletions and/or additions of at least one amino acid residue.

Specific biologically active fragment of a polypeptide according to the disclosure will be understood in particular as designating a specific polypeptide fragment, such as defined above, having at least one of the characteristics of polypeptides according to the disclosure, especially in that it is: capable of inducing an immunogenic reaction directed against a SARS-CoV-2; and/or capable of being recognized by a specific antibody of a polypeptide according to the disclosure; and/or capable of linking to a polypeptide or to a nucleotide sequence of SARS-CoV-2; and/or capable of exerting a physiological activity, even partial, such as, for example, a dissemination or structural (capsid) activity; and/or capable of modulating, of inducing or of inhibiting the expression of SARS-CoV-2 gene or one of its variants, and/or capable of modulating the replication cycle of SARS-CoV-2 in the cell and/or the host organism.

The polypeptide fragments according to the disclosure can correspond to isolated or purified fragments naturally present in a SARS-CoV-2 or correspond to fragments which can be obtained by cleavage of said polypeptide by a proteolytic enzyme, such as trypsin or chymotrypsin or collagenase, or by a chemical reagent, such as cyanogen bromide (CNBr) or alternatively by placing said polypeptide in a very acidic environment, for example at pH 2.5. Such polypeptide fragments can likewise just as easily be prepared by chemical synthesis, from hosts transformed by an expression vector according to the disclosure containing a nucleic acid allowing the expression of said fragments, placed under the control of appropriate regulation and/or expression elements.

“Modified polypeptide” of a polypeptide according to the disclosure is understood as designating a polypeptide obtained by genetic recombination or by chemical synthesis as will be described below, having at least one modification with respect to the normal sequence. These modifications will especially be able to bear on amino acids at the origin of a specificity, of pathogenicity and/or of virulence, or at the origin of the structural conformation, and of the capacity of membrane insertion of the polypeptide according to the disclosure. It will thus be possible to create polypeptides of equivalent, increased or decreased activity, and of equivalent, narrower, or wider specificity. Among the modified polypeptides, it is necessary to mention the polypeptides in which up to 5 amino acids can be modified, truncated at the N- or C-terminal end, or even deleted or added.

As is indicated, the modifications of the polypeptide will especially have as objective: to render it capable of modulating, of inhibiting or of inducing the expression of at least one SARS-CoV-2 gene and/or capable of modulating the replication cycle of SARS-CoV-2 in the cell and/or the host organism, of allowing its incorporation into vaccine compositions, and/or of modifying its bioavailability as a compound for therapeutic use.

The methods allowing said modulations on eukaryotic or prokaryotic cells to be demonstrated are well known to the person skilled in the art. It is likewise well understood that it will be possible to use the nucleotide sequences coding for said modified polypeptides for said modulations, for example through vectors according to the disclosure and described below, in order, for example, to prevent or to treat the pathologies linked to the infection.

The preceding modified polypeptides can be obtained by using combinatorial chemistry, in which it is possible to systematically vary parts of the polypeptide before testing them on models, cell cultures or microorganisms for example, to select the compounds which are most active or have the properties sought.

Chemical synthesis likewise has the advantage of being able to use unnatural amino acids, or nonpeptide bonds. Thus, in order to improve the duration of life of the polypeptides according to the disclosure, it may be of interest to use unnatural amino acids, for example in D form, or else amino acid analogs, especially sulfur-containing forms, for example.

Finally; it will be possible to integrate the structure of the polypeptides according to the disclosure, its specific or modified homologous forms, into chemical structures of polypeptide type or others. Thus, it may be of interest to provide at the N- and C-terminal ends compounds not recognized by the proteases.

The nucleotide sequences coding for a polypeptide according to the disclosure are likewise part of the disclosure.

The disclosure likewise relates to nucleotide sequences utilizable as a primer or probe, characterized in that said sequences are selected from the nucleotide sequences according to the disclosure.

The disclosure additionally relates to the use of a nucleotide sequence according to the disclosure as a primer or probe for the detection and/or the amplification of nucleic acid sequences.

The nucleotide sequences according to the disclosure can thus be used to amplify nucleotide sequences, especially by the PCR technique (polymerase chain reaction) (Erlich, 1989; Innis et al., 1990; Rolfs et al., 1991; and White et al., 1997). These oligodeoxyribonucleotide or oligoribonucleotide primers advantageously have a length of at least 8 nucleotides, preferably of at least 12 nucleotides, and even more preferentially at least 20 nucleotides. Other amplification techniques of the target nucleic acid can be advantageously employed as alternatives to PCR.

The nucleotide sequences of the disclosure, in particular the primers according to the disclosure, can likewise be employed in other procedures of amplification of a target nucleic acid, such as: the TAS technique (Transcription-based Amplification System), described by Kwoh et al. in 1989; the 3 SR technique (Self-Sustained Sequence Replication), described by Guatelli et al. in 1990; the NASBA technique (Nucleic Acid Sequence Based Amplification), described by Kievitis et al. in 1991; the SDA technique (Strand Displacement Amplification) (Walker et al., 1992); the TMA technique (Transcription Mediated Amplification).

The polynucleotides of the disclosure can also be employed in techniques of amplification or of modification of the nucleic acid serving as a probe, such as: the LCR technique (Ligase Chain Reaction), described by Landegren et al. in 1988 and improved by Barany et al. in 1991, which employs a thermostable ligase; the RCR technique (Repair Chain Reaction), described by Segev in 1992; the CPR technique (Cycling Probe Reaction), described by Duck et al. in 1990; the amplification technique with Q-beta replicase, described by Miele et al. in 1983 and especially improved by Chu et al. in 1986, Lizardi et al. in 1988, then by Burg et al. as well as by Stone et al. in 1996.

In the case where the target polynucleotide to be detected is possibly an RNA, for example an mRNA, it will be possible to use, prior to the employment of an amplification reaction with the aid of at least one primer according to the disclosure or to the employment of a detection procedure with the aid of at least one probe of the disclosure, an enzyme of reverse transcriptase type in order to obtain a cDNA from the RNA contained in the biological sample. The cDNA obtained will thus serve as a target for the primer(s) or the probe(s) employed in the amplification or detection procedure according to the disclosure.

The detection probe will be chosen in such a manner that it hybridizes with the target sequence or the amplicon generated from the target sequence. By way of sequence, such a probe will advantageously have a sequence of at least 12 nucleotides, in particular of at least 20 nucleotides, and preferably of at least 100 nucleotides.

The disclosure also comprises the nucleotide sequences utilizable as a probe or primer according to the disclosure, characterized in that they are labeled with a radioactive compound or with a nonradioactive compound. The unlabeled nucleotide sequences can be used directly as probes or primers, although the sequences are generally labeled with a radioactive element (³²P, ³⁵S, ³H, ¹²⁵I) or with a nonradioactive molecule (biotin, acetylaminofluorene, digoxigenin, 5-bromodeoxyuridine, fluorescein) to obtain probes which are utilizable for numerous applications. Examples of nonradioactive labeling of nucleotide sequences are described, for example, in French Patent No. 78.10975 or by Urdea et al. or by Sanchez-Pescador et al. in 1988. In the latter case, it will also be possible to use one of the labeling methods described in patents FR-2 422 956 and FR-2 518 755.

The hybridization technique can be carried out in various manners (Matthews et al., 1988). The most general method consists in immobilizing the nucleic acid extract of cells on a support (such as nitrocellulose, nylon, polystyrene) and in incubating, under well-defined conditions, the immobilized target nucleic acid with the probe. After hybridization, the excess of probe is eliminated and the hybrid molecules formed are detected by the appropriate method (measurement of the radioactivity, of the fluorescence or of the enzymatic activity linked to the probe).

The disclosure likewise comprises the nucleotide sequences according to the disclosure, characterized in that they are immobilized on a support, covalently or noncovalently.

According to another advantageous mode of employing nucleotide sequences according to the disclosure, the latter can be used immobilized on a support and can thus serve to capture, by specific hybridization, the target nucleic acid obtained from the biological sample to be tested. If necessary, the solid support is separated from the sample and the hybridization complex formed between said capture probe and the target nucleic acid is then detected with the aid of a second probe, a so-called detection probe, labeled with an easily detectable element.

Another subject of the present disclosure is a vector for the cloning and/or expression of a sequence, characterized in that it contains a nucleotide sequence according to the disclosure.

The vectors according to the disclosure, characterized in that they contain the elements allowing the expression in a determined host cell, are likewise part of the disclosure. The vector must then contain a promoter, signals of initiation and termination of translation, as well as appropriate regions of regulation of transcription. It must be able to be maintained stably in the host cell and can optionally have particular signals specifying the secretion of the translated protein. These different elements are chosen as a function of the host cell used. To this end, the nucleotide sequences according to the disclosure can be inserted into autonomous replication vectors within the chosen host, or integrated vectors of the chosen host. Such vectors will be prepared according to the methods currently used by the person skilled in the art, and it will be possible to introduce the clones resulting therefrom into an appropriate host by standard methods, such as, for example, lipofection, electroporation and thermal shock. The vectors according to the disclosure are, for example, vectors of plasmid or viral origin. A preferred vector for the expression of polypeptides of the disclosure is BPIV3c.

It is today possible to produce recombinant polypeptides in relatively large quantity by genetic engineering using the cells transformed by expression vectors according to the disclosure or using transgenic animals according to the disclosure. The procedures for preparation of a polypeptide of the disclosure in recombinant form, characterized in that they employ a vector and/or a cell transformed by a vector according to the disclosure and/or a transgenic animal comprising one of said transformed cells according to the disclosure, are themselves comprised in the present disclosure. Among said procedures for preparation of a polypeptide of the disclosure in recombinant form, the preparation procedures employing a vector, and/or a cell transformed by said vector and/or a transgenic animal comprising one of said transformed cells, containing a nucleotide sequence according to the disclosure coding for a polypeptide of SARS-CoV-2, are preferred. The recombinant polypeptides obtained as indicated above can just as well be present in glycosylated form as in nonglycosylated form and can or cannot have the natural tertiary structure.

More particularly, the disclosure relates to a procedure for preparation of a polypeptide of the disclosure comprising the following steps: a) culture of transformed cells under conditions allowing the expression of a recombinant polypeptide by a nucleotide sequence according to the disclosure; b) if need be, recovery of said recombinant polypeptide.

When the procedure for preparation of a polypeptide of the disclosure employs a transgenic animal according to the disclosure, the recombinant polypeptide is then extracted from said animal.

The disclosure also relates to a polypeptide which is capable of being obtained by a procedure of the disclosure such as described previously.

The disclosure also comprises a procedure for preparation of a synthetic polypeptide, characterized in that it uses a sequence of amino acids of polypeptides according to the disclosure. The disclosure likewise relates to a synthetic polypeptide obtained by a procedure according to the disclosure.

The polypeptides according to the disclosure can likewise be prepared by techniques which are conventional in the field of the synthesis of peptides. This synthesis can be carried out in homogeneous solution or in solid phase. For example, reference can be made to the technique of synthesis in homogeneous solution described by Houben-Weyl in 1974. This method of synthesis consists in successively condensing, two by two, the successive amino acids in the order required, or in condensing amino acids and fragments formed previously and already containing several amino acids in the appropriate order, or alternatively several fragments previously prepared in this way, it being understood that it will be necessary to protect beforehand all the reactive functions carried by these amino acids or fragments, with the exception of amine functions of one and carboxyls of the other or vice-versa, which must normally be involved in the formation of peptide bonds, especially after activation of the carboxyl function, according to the methods well known in the synthesis of peptides. According to another preferred technique of the disclosure, recourse will be made to the technique described by Merrifield. To make a peptide chain according to the Merrifield procedure, recourse is made to a very porous polymeric resin, on which is immobilized the first C-terminal amino acid of the chain. This amino acid is immobilized on a resin through its carboxyl group and its amine function is protected. The amino acids which are going to form the peptide chain are thus immobilized, one after the other, on the amino group, which is deprotected beforehand each time, of the portion of the peptide chain already formed, and which is attached to the resin. When the whole of the desired peptide chain has been formed, the protective groups of the different amino acids forming the peptide chain are eliminated and the peptide is detached from the resin with the aid of an acid.

The disclosure additionally relates to hybrid polypeptides having at least one polypeptide according to the disclosure, and a sequence of a polypeptide capable of inducing an immune response in man or animals.

Advantageously, the antigenic determinant is such that it is capable of inducing a humoral and/or cellular response. It will be possible for such a determinant to comprise a polypeptide according to the disclosure in glycosylated form used with a view to obtaining immunogenic compositions capable of inducing the synthesis of antibodies directed against multiple epitopes. Said polypeptides or their glycosylated fragments are likewise part of the disclosure. These hybrid molecules can be formed, in part, of a polypeptide carrier molecule or of fragments thereof according to the disclosure, associated with a possibly immunogenic part, in particular an epitope of the diphtheria toxin, the tetanus toxin, a surface antigen of the hepatitis B virus (patent FR 79 21811), the VP1 antigen of the poliomyelitis virus or any other viral or bacterial toxin or antigen. The procedures for synthesis of hybrid molecules encompass the methods used in genetic engineering for constructing hybrid nucleotide sequences coding for the polypeptide sequences sought. It will be possible, for example, to refer advantageously to the technique for obtainment of genes coding for fusion proteins described by Minton in 1984. Said hybrid nucleotide sequences coding for a hybrid polypeptide as well as the hybrid polypeptides according to the disclosure characterized in that they are recombinant polypeptides obtained by the expression of said hybrid nucleotide sequences are likewise part of the disclosure.

The disclosure likewise comprises the vectors characterized in that they contain one of said hybrid nucleotide sequences. The host cells transformed by said vectors, the transgenic animals comprising one of said transformed cells as well as the procedures for preparation of recombinant polypeptides using said vectors, said transformed cells and/or said transgenic animals are, of course, likewise part of the disclosure.

The polypeptides according to the disclosure, the antibodies according to the disclosure described below and the nucleotide sequences according to the disclosure can advantageously be employed in procedures for the detection and/or identification of SARS-CoV-2 in a biological sample (biological tissue or fluid) capable of containing them. These procedures, according to the specificity of the polypeptides, the antibodies and the nucleotide sequences according to the disclosure which will be used, will in particular be able to detect and/or to identify a SARS-CoV-2.

The polypeptides according to the disclosure can advantageously be employed in a procedure for the detection and/or the identification of SARS-CoV-2 in a biological sample (biological tissue or fluid) capable of containing them, characterized in that it comprises the following steps: a) contacting of this biological sample with a polypeptide or one of its fragments according to the disclosure (under conditions allowing an immunological reaction between said polypeptide and the antibodies possibly present in the biological sample); and b) demonstration of the antigen-antibody complexes possibly formed. Preferably, the biological sample is formed by a fluid, for example whole blood or biopsies. Any conventional procedure can be employed for carrying out such a detection of the antigen-antibody complexes possibly formed. By way of example, a preferred method brings into play immunoenzymatic processes according to the ELISA technique, by immunofluorescence, or radioimmunological processes (RIA) or their equivalent.

Thus, the disclosure likewise relates to the polypeptides according to the disclosure, labeled with the aid of an adequate label such as of the enzymatic, fluorescent or radioactive type. Such methods comprise, for example, the following steps: 1) deposition of determined quantities of a polypeptide composition according to the disclosure in the wells of a microtiter plate; 2) introduction into said wells of increasing dilutions of serum, or of a biological sample other than that defined previously, having to be analyzed; 3) incubation of the microplate; and 4) introduction into the wells of the microtiter plate of labeled antibodies directed against mammal immunoglobulins, the labeling of these antibodies having been carried out with the aid of an enzyme selected from those which are capable of hydrolyzing a substrate by modifying the absorption of the radiation of the latter, at least at a determined wavelength, for example at 550 nm, detection, by comparison with a control test, of the quantity of hydrolyzed substrate.

The disclosure likewise relates to a kit or set for the detection and/or identification of SARS-CoV-2, characterized in that it comprises the following elements: 1) a polypeptide according to the disclosure; 2) if need be, the reagents for the formation of the medium favorable to the immunological or specific reaction; 3) if need be, the reagents allowing the detection of the antigen-antibody complexes produced by the immunological reaction between the polypeptide(s) of the disclosure and the antibodies possibly present in the biological sample, these reagents likewise being able to carry a label, or to be recognized in their turn by a labeled reagent, more particularly in the case where the polypeptide according to the disclosure is not labeled; 4) if need be, a biological reference sample (negative control) devoid of antibodies recognized by a polypeptide according to the disclosure; and 5) if need be, a biological reference sample (positive control) containing a predetermined quantity of antibodies recognized by a polypeptide according to the disclosure.

The polypeptides according to the disclosure allow monoclonal or polyclonal antibodies to be prepared which are characterized in that they specifically recognize the polypeptides according to the disclosure. It will advantageously be possible to prepare the monoclonal antibodies from hybridomas according to the technique described by Kohler and Milstein in 1975. It will be possible to prepare the polyclonal antibodies, for example, by immunization of an animal, in particular a mouse, with a polypeptide or a DNA, according to the disclosure, associated with an adjuvant of the immune response, and then purification of the specific antibodies contained in the serum of the immunized animals on an affinity column on which the polypeptide which has served as an antigen has previously been immobilized. The polyclonal antibodies according to the disclosure can also be prepared by purification, on an affinity column on which a polypeptide according to the disclosure has previously been immobilized, of the antibodies contained in the serum of animals infected by a SARS-CoV-2.

The disclosure likewise relates to mono- or polyclonal antibodies or their fragments, or chimeric antibodies, characterized in that they are capable of specifically recognizing a polypeptide according to the disclosure. It will likewise be possible for the antibodies of the disclosure to be labeled in the same manner as described previously for the nucleic probes of the disclosure, such as a labeling of enzymatic, fluorescent or radioactive type.

The disclosure is additionally directed at a procedure for the detection and/or identification of SARS-CoV-2, in a biological sample, characterized in that it comprises the following steps: a) contacting of the biological sample (biological tissue or fluid) with a mono- or polyclonal antibody according to the disclosure (under conditions allowing an immunological reaction between said antibodies and the polypeptides of SARS-CoV-2, including RBD, possibly present in the biological sample); and b) demonstration of the antigen-antibody complex possibly formed.

Likewise within the scope of the disclosure is a kit or set for the detection and/or the identification of SARS-CoV-2, characterized in that it comprises the following components: a) a polyclonal or monoclonal antibody according to the disclosure, if need be labeled; b) if need be, a reagent for the formation of the medium favorable to the carrying out of the immunological reaction; c) if need be, a reagent allowing the detection of the antigen-antibody complexes produced by the immunological reaction, this reagent likewise being able to carry a label, or being capable of being recognized in its turn by a labeled reagent, more particularly in the case where said monoclonal or polyclonal antibody is not labeled; and d) if need be, reagents for carrying out the lysis of cells of the sample tested.

The present disclosure likewise relates to a procedure for the detection and/or the identification of SARS-CoV-2 in a biological sample, characterized in that it employs a nucleotide sequence according to the disclosure. More particularly, the disclosure relates to a procedure for the detection and/or the identification of SARS-CoV-2, in a biological sample, characterized in that it contains the following steps: a) if need be, isolation of the DNA from the biological sample to be analyzed; b) specific amplification of the DNA of the sample with the aid of at least one primer, or a pair of primers, according to the disclosure; and c) demonstration of the amplification products. These can be detected, for example, by the technique of molecular hybridization utilizing a nucleic probe according to the disclosure. This probe will advantageously be labeled with a nonradioactive (cold probe) or radioactive element.

For the purposes of the present disclosure, “DNA of the biological sample” or “DNA contained in the biological sample” will be understood as meaning either the DNA present in the biological sample considered, or possibly the cDNA obtained after the action of an enzyme of reverse transcriptase type on the RNA present in said biological sample.

Another aim of the present disclosure consists in a procedure according to the disclosure, characterized in that it comprises the following steps: a) contacting of a nucleotide probe according to the disclosure with a biological sample, the DNA contained in the biological sample having, if need be, previously been made accessible to hybridization under conditions allowing the hybridization of the probe with the DNA of the sample; and b) demonstration of the hybrid formed between the nucleotide probe and the DNA of the biological sample.

The present disclosure also relates to a procedure according to the disclosure, characterized in that it comprises the following steps: a) contacting of a nucleotide probe immobilized on a support according to the disclosure with a biological sample, the DNA of the sample having, if need be, previously been made accessible to hybridization, under conditions allowing the hybridization of the probe with the DNA of the sample; b) contacting of the hybrid formed between the nucleotide probe immobilized on a support and the DNA contained in the biological sample, if need be after elimination of the DNA of the biological sample which has not hybridized with the probe, with a nucleotide probe labeled according to the disclosure; and c) demonstration of the novel hybrid formed in step b). According to an advantageous embodiment of the procedure for detection and/or identification defined previously, this is characterized in that, prior to step a), the DNA of the biological sample is first amplified with the aid of at least one primer according to the disclosure.

The disclosure is additionally directed at a kit or set for the detection and/or the identification of SARS-CoV-2, characterized in that it comprises the following elements: a) a nucleotide probe according to the disclosure; b) if need be, the reagents necessary for the carrying out of a hybridization reaction; and c) if need be, at least one primer according to the disclosure as well as the reagents necessary for an amplification reaction of the DNA.

The disclosure likewise relates to a kit or set for the detection and/or the identification of SARS-CoV-2 characterized in that it comprises the following components: a) a nucleotide probe, called a capture probe, according to the disclosure; b) an oligonucleotide probe, called a revealing probe, according to the disclosure, and c) if need be, at least one primer according to the disclosure, as well as the reagents necessary for an amplification reaction of the DNA.

The disclosure also relates to a kit or set for the detection and/or identification of SARS-CoV-2, characterized in that it comprises the following elements: a) at least one primer according to the disclosure; b) if need be, the reagents necessary for carrying out a DNA amplification reaction; and c) if need be, a component allowing the sequence of the amplified fragment to be verified, more particularly an oligonucleotide probe according to the disclosure.

The disclosure additionally relates to the use of a nucleotide sequence according to the disclosure, of a polypeptide according to the disclosure, of an antibody according to the disclosure, of a cell according to the disclosure, and/or of an animal transformed according to the disclosure, for the selection of an organic or inorganic compound capable of modulating, inducing or inhibiting the expression of genes, and/or of modifying the cellular replication of SARS-CoV-2 or capable of inducing or of inhibiting the pathologies including reducing the incidence of and severity of clinical signs linked to an infection by a SARS-CoV-2.

The disclosure likewise comprises a method of selection of compounds capable of binding to a polypeptide or one of its fragments according to the disclosure, capable of binding to a nucleotide sequence according to the disclosure, or capable of recognizing an antibody according to the disclosure, and/or capable of modulating, inducing or inhibiting the expression of genes, and/or of modifying the cellular replication of SARS-CoV-2 or capable of inducing or inhibiting the pathologies including reducing the incidence of and severity of clinical signs linked to an infection by a SARS-CoV-2, characterized in that it comprises the following steps: a) contacting of said compound with said polypeptide, said nucleotide sequence, or with a cell transformed according to the disclosure and/or administration of said compound to an animal transformed according to the disclosure; and b) determination of the capacity of said compound to bind to said polypeptide or said nucleotide sequence, or to modulate, induce or inhibit the expression of genes, or to modulate the growth or the replication of SARS-CoV-2, or to induce or inhibit in said transformed animal the pathologies linked to an infection by SARS-CoV-2 (designated activity of said compound). The compounds capable of being selected can be organic compounds such as polypeptides or carbohydrates or any other organic or inorganic compounds already known, or novel organic compounds elaborated by molecular modelling techniques and obtained by chemical or biochemical synthesis, these techniques being known to the person skilled in the art. It will be possible to use said selected compounds to modulate the cellular replication of SARS-CoV-2 and thus to control infection by this virus, the methods allowing said modulations to be determined being well known to the person skilled in the art. This modulation can be carried out, for example, by an agent capable of binding to a protein and thus of inhibiting or of potentiating its biological activity, or capable of binding to an envelope protein of the external surface of said virus and of blocking the penetration of said virus into the host cell or of favoring the action of the immune system of the infected organism directed against said virus. This modulation can likewise be carried out by an agent capable of binding to a nucleotide sequence of a DNA of said virus and of blocking, for example, the expression of a polypeptide whose biological or structural activity is necessary for the replication or for the proliferation of said virus host cells to host cells in the host animal.

The disclosure relates to the compounds capable of being selected by a selection method according to the disclosure.

The disclosure likewise relates to a pharmaceutical composition comprising a compound selected from the following compounds: a) a nucleotide sequence according to the disclosure; b) a polypeptide according to the disclosure; c) a vector, a viral particle or a cell transformed according to the disclosure; d) an antibody according to the disclosure; and e) a compound capable of being selected by a selection method according to the disclosure; possibly in combination with a pharmaceutically acceptable carrier and, if need be, with one or more adjuvants of the appropriate immunity.

The disclosure also relates to an immunogenic and/or vaccine composition, characterized in that it comprises a compound selected from the following compounds: a) a nucleotide sequence according to the disclosure; b) a polypeptide according to the disclosure; c) a vector or a viral particle according to the disclosure; and d) a cell according to the disclosure.

In one embodiment, the vaccine composition according to the disclosure is characterized in that it comprises a mixture of at least two of said compounds a), b), c) and d) above and in that one of the two said compounds is related to the SARS-CoV-2.

In another embodiment of the disclosure, the vaccine composition is characterized in that it comprises at least one compound a), b), c), or d) above which is related to SARS-CoV-2. In still another embodiment, the vaccine composition is characterized in that it comprises at least one compound a), b), c), or d) above which is related to SARS-CoV-2 RBD.

A compound related to the SARS-CoV-2 is understood here as respectively designating a compound obtained from the genomic sequence of the SARS-CoV-2 and/or RBD of SARS-CoV-2.

The disclosure is additionally aimed at an immunogenic and/or vaccine composition, characterized in that it comprises at least one of the following compounds: 1) a nucleotide sequence encoding SEQ ID No. 3 or one of its fragments or homologues; 2) a polypeptide of sequence SEQ ID No. 1, SEQ ID No. 3, or SEQ ID No. 12 or one of their fragments, or a modification thereof; 3) a vector or a viral particle comprising a nucleotide sequence encoding SEQ ID No. 3 or one of its fragments or homologues; 4) a transformed cell capable of expressing a polypeptide of sequence SEQ ID No. 3, or one of its fragments, or a modification thereof; or 5) a mixture of at least two of said compounds.

The disclosure also comprises an immunogenic and/or vaccine composition according to the disclosure, characterized in that it comprises said mixture of at least two of said compounds as a combination product for simultaneous, separate or protracted use for the prevention or the treatment of infection by a SARS-CoV-2.

The disclosure is likewise directed at a pharmaceutical composition according to the disclosure, for the prevention or the treatment of an infection by a SARS-CoV-2.

It is understood that “prevention” as used in the present disclosure, includes the complete prevention of infection by a SARS-CoV-2, but also encompasses a reduction in the severity of or incidence of clinical signs associated with or caused by SARS-CoV-2 infection. Such prevention is also referred to herein as a protective effect.

The disclosure likewise concerns the use of a composition according to the disclosure, for the preparation of a medicament intended for the prevention or the treatment of infection by a SARS-CoV-2.

Under another aspect, the disclosure relates to a vector, a viral particle or a cell according to the disclosure, for the treatment and/or the prevention of a disease by gene therapy.

Finally, the disclosure comprises the use of a vector, of a viral particle or of a cell according to the disclosure for the preparation of a medicament intended for the treatment and/or the prevention of a disease by gene therapy.

The polypeptides of the disclosure entering into the immunogenic or vaccine compositions according to the disclosure can be selected by techniques known to the person skilled in the art such as, for example, depending on the capacity of said polypeptides to stimulate the T cells, which is translated, for example, by their proliferation or the secretion of interleukins, and which leads to the production of antibodies directed against said polypeptides.

In pigs, as in mice, in which a weight dose of the vaccine composition comparable to the dose used in man is administered, the antibody reaction is tested by taking of the serum followed by a study of the formation of a complex between the antibodies present in the serum and the antigen of the vaccine composition, according to the usual techniques.

The pharmaceutical compositions according to the disclosure will contain an effective quantity of the compounds of the disclosure, that is to say in sufficient quantity of said compound(s) allowing the desired effect to be obtained, such as, for example, the modulation of the cellular replication of SARS-CoV-2. The person skilled in the art will know how to determine this quantity, as a function, for example, of the age and of the weight of the individual to be treated, of the state of advancement of the pathology, of the possible secondary effects and by means of a test of evaluation of the effects obtained on a population range, these tests being known in these fields of application.

According to the disclosure, said vaccine combinations will preferably be combined with a pharmaceutically or veterinary acceptable carrier and, if need be, with one or more adjuvants of the appropriate immunity.

Today, various types of vaccines are available for protecting animals or man against infectious diseases: attenuated living microorganisms (M. bovis—BCG for tuberculosis), inactivated microorganisms (influenza virus), a cellular extracts (Bordetella pertussis for whooping cough), recombinant proteins (surface antigen of the hepatitis B virus), polysaccharides (pneumococcal). Vaccines prepared from synthetic peptides or genetically modified microorganisms expressing heterologous antigens are in the course of experimentation. More recently still, recombined plasmid DNAs carrying genes coding for protective antigens have been proposed as an alternative vaccine strategy. This type of vaccination is carried out with a particular plasmid originating from a plasmid of E. coli which does not replicate in vivo and which codes uniquely for the vaccinating protein. Animals have been immunized by simply injecting the naked plasmid DNA into the muscle. This technique leads to the expression of the vaccine protein in situ and to an immune response of cellular type (CTL) and of humoral type (antibody). This double induction of the immune response is one of the principal advantages of the vaccination technique with naked DNA.

The constitutive nucleotide sequence of the vaccine composition according to the disclosure can be injected into the host after having been coupled to compounds which favor the penetration of this polynucleotide into the interior of the cell or its transport to the cell nucleus. The resultant conjugates can be encapsulated in polymeric microparticles, as described in the international application No. WO 94/27238 (Medisorb Technologies International).

According to another embodiment of the vaccine composition according to the disclosure, the nucleotide sequence, preferably a DNA, is complexed with DEAE-dextran (Pagano et al., 1967) or with nuclear proteins (Kaneda et al., 1989), with lipids (Felgner et al., 1987) or encapsulated in liposomes (Fraley et al., 1980) or else introduced in the form of a gel facilitating its transfection into the cells (Midoux et al., 1993, Pastore et al., 1994). The polynucleotide or the vector according to the disclosure can also be in suspension in a buffer solution or be combined with liposomes.

Advantageously, such a vaccine will be prepared according to the technique described by Tacson et al. or Huygen et al. in 1996 or alternatively according to the technique described by Davis et al. in the international application No. WO 95/11307.

Such a vaccine can likewise be prepared in the form of a composition containing a vector according to the disclosure, placed under the control of regulation elements allowing its expression in man or animal. It will be possible, for example, to use, by way of in vivo expression vector of the polypeptide antigen of interest, the plasmid pcDNA3 or the plasmid pcDNA1/neo, both marketed by Invitrogen (R&D Systems, Abingdon, United Kingdom). It is also possible to use the plasmid VlJns.tPA, described by Shiver et al. in 1995. Such a vaccine will advantageously comprise, apart from the recombinant vector, a saline solution, for example a sodium chloride solution.

As far as the vaccine formulations are concerned, these can comprise adjuvants of the appropriate immunity which are known to the person skilled in the art, such as, for example, those described above.

These compounds can be administered by the systemic route, in particular by the intravenous route, by the intramuscular, intradermal or subcutaneous route, or by the oral route. In a more preferred manner, the vaccine composition comprising polypeptides according to the disclosure will be administered by the intramuscular route, through the food or by nebulization several times, staggered over time.

Their administration modes, dosages and optimum pharmaceutical forms can be determined according to the criteria generally taken into account in the establishment of a treatment adapted to an animal such as, for example, the age or the weight, the seriousness of its general condition, the tolerance to the treatment and the secondary effects noted. Preferably, the vaccine of the present disclosure is administered in an amount that is protective or provides a protective effect against SARS-CoV-2 infection.

For example, in the case of a vaccine according to the present disclosure comprising a polypeptide encoded by a nucleotide sequence of the genome of SARS-CoV-2, or a homologue or fragment thereof, the polypeptide will be administered one time or several times, spread out over time, directly or by means of a transformed cell capable of expressing the polypeptide, in an amount of about 0.1 to 10 μg per kilogram weight of the animal, preferably about 0.2 to about 5 μg/kg, more preferably about 0.5 to about 2 μg/kg for a dose.

The present disclosure likewise relates to the use of nucleotide sequences of SARS-CoV-2 according to the disclosure for the construction of autoreplicative retroviral vectors and the therapeutic applications of these, especially in the field of gene therapy in vivo.

The feasibility of gene therapy applied to man no longer needs to be demonstrated and this relates to numerous therapeutic applications like genetic diseases, infectious diseases and cancers. Numerous documents of the prior art describe the means of employing gene therapy, especially through viral vectors. Generally speaking, the vectors are obtained by deletion of at least some of the viral genes which are replaced by the genes of therapeutic interest. Such vectors can be propagated in a complementation cell line which supplies in trans the deleted viral functions in order to generate a defective viral vector particle for replication but capable of infecting a host cell. To date, the retroviral vectors are amongst the most widely used and their mode of infection is widely described in the literature accessible to the person skilled in the art.

The principle of gene therapy is to deliver a functional gene, called a gene of interest, of which the RNA or the corresponding protein will produce the desired biochemical effect in the targeted cells or tissues. On the one hand, the insertion of genes allows the prolonged expression of complex and unstable molecules such as RNAs or proteins which can be extremely difficult or even impossible to obtain or to administer directly. On the other hand, the controlled insertion of the desired gene into the interior of targeted specific cells allows the expression product to be regulated in defined tissues. For this, it is necessary to be able to insert the desired therapeutic gene into the interior of chosen cells and thus to have available a method of insertion capable of specifically targeting the cells or the tissues chosen. Some preferred genes of interest for the present disclosure are those that encode RBD.

Among the methods of insertion of genes, such as, for example, microinjection, especially the injection of naked plasmid DNA, electroporation, homologous recombination, the use of viral particles, such as retroviruses, is widespread. However, applied in vivo, the gene transfer systems of recombinant retroviral type at the same time have a weak infectious power (insufficient concentration of viral particles) and a lack of specificity with regard to chosen target cells.

The production of cell-specific viral vectors, having a tissue-specific tropism, and whose gene of interest can be translated adequately by the target cells, is realizable, for example, by fusing a specific ligand of the target host cells to the N-terminal part of a surface protein of the envelope of SARS-CoV-2. It is possible to mention, for example, the construction of retroviral particles having the CD4 molecule on the surface of the envelope so as to target the human cells infected by the HIV virus, viral particles having a peptide hormone fused to an envelope protein to specifically infect the cells expressing the corresponding receptor or else alternatively viral particles having a fused polypeptide capable of immobilizing on the receptor of the epidermal growth factor (EGF). In another approach, single-chain fragments of antibodies directed against surface antigens of the target cells are inserted by fusion with the N-terminal part of the envelope protein.

For the purposes of the present disclosure, a gene of interest in use in the disclosure can be obtained from a eukaryotic or prokaryotic organism or from a virus by any conventional technique. It is, preferably, capable of producing an expression product having a therapeutic effect and it can be a product homologous to the cell host or, alternatively, heterologous. In the scope of the present disclosure, a gene of interest can code for an (1) intracellular or (2) membrane product present on the surface of the host cell or (3) secreted outside the host cell. It can therefore comprise appropriate additional elements such as, for example, a sequence coding for a secretion signal. These signals are known to the person skilled in the art.

In accordance with the aims pursued by the present disclosure, a gene of interest can code for a protein corresponding to all or part of a native protein as found in nature. It can likewise be a chimeric protein, for example arising from the fusion of polypeptides of various origins or from a mutant having improved and/or modified biological properties. Such a mutant can be obtained, by conventional biological techniques, by substitution, deletion and/or addition of one or more amino acid residues.

The disclosure thus relates to the vectors characterized in that they comprise a nucleotide sequence of SARS-CoV-2 according to the disclosure, and in that they additionally comprise a gene of interest.

The present disclosure likewise relates to viral particles generated from said vector according to the disclosure. It additionally relates to methods for the preparation of viral particles according to the disclosure, characterized in that they employ a vector according to the disclosure, including viral pseudoparticles (VLP, virus-like particles).

The disclosure likewise relates to animal cells transfected by a vector according to the disclosure. Likewise comprised in the disclosure are animal cells, especially mammalian, infected by a viral particle according to the disclosure.

Additional genetically engineered vaccines, which are desirable in the present disclosure, are produced by techniques known in the art. Such techniques involve, but are not limited to, further manipulation of recombinant DNA, modification of or substitutions to the amino acid sequences of the recombinant proteins and the like. Genetically engineered vaccines based on recombinant DNA technology are made, for instance, by identifying alternative portions of the viral gene encoding proteins responsible for inducing a stronger immune or protective response in animals (e.g., proteins derived from the RBD). Such identified genes or immuno-dominant fragments can be cloned into standard protein expression vectors, such as the baculovirus vector, and used to infect appropriate host cells. The host cells are cultured, thus expressing the desired vaccine proteins, which can be purified to the desired extent and formulated into a suitable vaccine product.

If the clones retain any undesirable natural abilities of causing disease, it is also possible to pinpoint the nucleotide sequences in the viral genome responsible for any residual virulence, and genetically engineer the virus avirulent through, for example, site-directed mutagenesis. Site-directed mutagenesis is able to add, delete or change one or more nucleotides. An oligonucleotide is synthesized containing the desired mutation and annealed to a portion of single stranded viral DNA. The hybrid molecule, which results from that procedure, is employed to transform bacteria. Then double-stranded DNA, which is isolated containing the appropriate mutation, is used to produce full-length DNA by ligation to a restriction fragment of the latter that is subsequently transfected into a suitable cell culture. Ligation of the genome into the suitable vector for transfer may be accomplished through any standard technique known to those of ordinary skill in the art. Transfection of the vector into host cells for the production of viral progeny may be done using any of the conventional methods such as calcium-phosphate or DEAE-dextran mediated transfection, electroporation, protoplast fusion and other well-known techniques. The cloned virus then exhibits the desired mutation. Alternatively, two oligonucleotides can be synthesized which contain the appropriate mutation. These may be annealed to form double-stranded DNA that can be inserted in the viral DNA to produce full-length DNA.

Genetically engineered proteins, useful in vaccines, for instance, may be expressed in insect cells, yeast cells or mammalian cells. The genetically engineered proteins, which may be purified or isolated by conventional methods, can be directly inoculated into animals to confer protection against viral infection caused by SARS-CoV-2. An insect cell line (like HI-FIVE) can be transformed with a transfer vector containing nucleic acid molecules obtained from the virus or copied from the viral genome which encodes one or more of the immuno-dominant proteins of the virus. The transfer vector includes, for example, linearized baculovirus DNA and a plasmid containing the desired polynucleotides. The host cell line may be co-transfected with the linearized baculovirus DNA and a plasmid in order to make a recombinant baculovirus.

An immunologically effective amount of one of the vaccines or immunogenic compositions of the present disclosure is administered to an animal in need of protection against viral infection, pneumonia, fever, cough, and loss of taste and/or smell. The immunologically effective amount or the immunogenic amount that inoculates the animal can be easily determined or readily titrated by routine testing. An effective amount is one in which a sufficient immunological response to the vaccine is attained to protect the animal exposed to SARS-CoV-2. Preferably, the animal receiving a dose of a vaccine or immunogenic composition according to this disclosure is protected to an extent in which one to all of the adverse physiological symptoms or effects of the viral disease are significantly reduced, ameliorated or totally prevented.

The vaccine can be administered in a single dose or in repeated doses with single doses being preferred. Single dose vaccines provide protection after a single dose without the need for any booster or subsequent dosages. Protection can include the complete prevention of clinical signs of infection, or a lessening of the severity, duration, or likelihood of the manifestation of one or more clinical signs of infection.

Desirably, the vaccine is administered to an animal not yet exposed to the SARS-CoV-2 virus. When administered as a liquid, the present vaccine may be prepared in the form of an aqueous solution, syrup, an elixir, a tincture and the like. Such formulations are known in the art and are typically prepared by dissolution of the antigen and other typical additives in the appropriate carrier or solvent systems. Suitable carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives that can be employed in the present vaccine include, but are not limited to, dextrose, conventional antioxidants and conventional chelating agents such as ethylenediamine tetraacetic acid (EDTA). Parenteral dosage forms must also be sterilized prior to use.

It should be appreciated that all scientific and technological terms used herein have the same meaning as commonly understood by those of ordinary skill in the art.

Another aspect of the present disclosure is the preparation of the combination vaccine(s) or immunogenic compositions. Such combinations can be between the different vaccine components described herein. For example, a vaccine of the present disclosure can include both protein portions and DNA portions of SARS-CoV-2, as described herein, which are administered concurrently or separately. Additionally, the combinations can be between the SARS-CoV-2 vaccine components described herein and antigens of other disease-causing organisms, such as those described above.

According to a further aspect, the vaccine or immunogenic composition is first dehydrated. If the composition is first lyophilized or dehydrated by other methods, then, prior to vaccination, said composition is rehydrated in aqueous (e.g. saline, PBS (phosphate buffered saline)) or non-aqueous solutions (e.g. oil emulsion (mineral oil, or vegetable/metabolizable oil based/single or double emulsion based), aluminum-based, carbomer based adjuvant).

The composition according to the disclosure may be applied intradermally, intratracheally, or intravaginally. The composition preferably may be applied intramuscularly or intranasally. In an animal body, it can prove advantageous to apply the pharmaceutical compositions as described above via an intravenous injection or by direct injection into target tissues. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the disclosure may be administered once or several times, also intermittently, for instance on a daily basis for several days, weeks or months, and in different dosages.

In a further aspect of the disclosure, a method for protecting humans from SARS-CoV-2 infection transmitted by animals is provided. In general, the method comprises administering at least one dose of a composition comprising the RBD protein from SARS-CoV-2 to a susceptible animal. Preferred susceptible animals are provided above. Such administration prevents or reduces the transmission from an infected animal to humans. In preferred forms, the transmission to humans will be reduced by at least 10, 20, 30, 40, 50, 60, 70, 80, 90, 95, or even 100%. Such a method removes a potential host or reservoir for SARS-CoV-2 and reduces the overall circulation of SARS-CoV-2. Preferred compositions are selected from those disclosed herein.

In a further aspect of the disclosure, a method of inducing a immunological response against SARS-CoV-2 is provided. In general, the method comprises the steps of administering a composition selected from the group consisting of a composition comprising at least one SARS-CoV-2 RDB protein and a pharmaceutical-acceptable carrier selected from the group consisting of a solvent, a dispersion media, a coating, a stabilizing agent, a diluent, a preservative, an antimicrobial agent, an antifungal agent, an isotonic agent, and an adsorption delaying agent; or a composition comprising at least one SARS-CoV-2 nucleotide sequence selected from the group consisting of the nucleotide sequence encoding the RBD of SARS-CoV-2; or any combination of these compositions, together with a pharmaceutical-acceptable carrier selected from the group consisting of a solvent, a dispersion media, a coating, a stabilizing agent, a diluent, a preservative, an antibacterial agent, an antifungal agent, an isotonic agent, and an adsorption delaying agent, to an animal in need thereof. In some forms, the RBD protein has at least 90% sequence homology with SEQ ID NO. 3. In some forms, the pharmaceutical-acceptable carrier comprises a stabilizing agent and/or a preservative and/or an antimicrobial agent. In some forms, the protein is present in the final composition in an amount from 0.2 to about 400 μg/ml. In some forms, the composition further comprises an immune stimulant. In some forms, the composition further comprises at least one immunological active component against another disease-causing organism. In some forms, the animal in need thereof is selected from the group consisting of humans, dogs, cats, ferrets, bats, cattle, camels, hamsters, horses, chimps, gorillas, anteaters, dolphins, alligators, and sheep. In some forms, the composition is administered a first time and a second time. In some forms, the second time is at least 10 days after the first time.

In another aspect of the disclosure, a composition comprising at least one BPI3Vc-vectored SARS-CoV-2 RBD protein and a pharmaceutical-acceptable carrier selected from the group consisting of a solvent, a dispersion media, a coating, a stabilizing agent, a diluent, a preservative, an antimicrobial agent, an antifungal agent, an isotonic agent, and an adsorption delaying agent, is provided. In some forms, the protein has at least 90, 91, 92, 93, 94, 95, 96, 97, 98, 99, 99.5, 99.6, 99.7, 99.8, 99.9, or even 100% sequence homology with SEQ ID NO. 3. In some forms, the pharmaceutical-acceptable carrier comprises a stabilizing agent and/or a preservative and/or an antimicrobial agent. In some forms, the protein is present in the final composition in an amount from 0.2 to about 400 μg/ml. In some forms, the composition further includes an immune stimulant. In some forms, the composition further comprises at least one immunological active component against another disease-causing organism. In some forms, the BPI3Vc-vectored SARS-CoV-2 RBD protein is encoded by a BPI3Vc vector having a sequence with at least 90, 91, 92, 93, 94, 95, 96, 97, 98, 99, 99.5, 99.6, 99.7, 99.8, 99.9, or even 100% sequence homology with SEQ ID NO. 13. In some forms, the vector includes an inserted sequence having at least 90% sequence homology with SEQ ID NO. 1. In some forms, the inserted sequence includes a sequence encoding an RBD protein having at least 90% sequence homology with SEQ ID NO. 3.

In another aspect of the disclosure, a composition comprising a vector comprising a BPI3Vc backbone and an inserted sequence is provided, wherein said inserted sequence includes a SARS-CoV-2 RBD coding sequence. In some forms, the BPI3Vc backbone comprises a sequence having at least 90% sequence homology with SEQ ID NO. 13. In some forms, the composition further comprises a pharmaceutical-acceptable carrier selected from the group consisting of a solvent, a dispersion media, a coating, a stabilizing agent, a diluent, a preservative, an antibacterial agent, an antifungal agent, an isotonic agent, and an adsorption delaying agent. In some forms, the SARS-CoV-2 RBD coding sequence encodes a sequence having at least 90, 91, 92, 93, 94, 95, 96, 97, 98, 99, 99.5, 99.6, 99.7, 99.8, 99.9, or even 100% sequence homology with SEQ ID NO. 3. In some forms, the SARS-CoV-2 RBD coding sequence is inserted between the N and P portions of the BPI3Vc backbone. In some forms, the pharmaceutical-acceptable carrier comprises a stabilizing agent and/or a preservative and/or an antimicrobial agent. In some forms, the nucleotide sequence is present in the final composition in an amount from 0.2 to about 400 μg/ml. In some forms, the composition further comprises an immune stimulant. In some forms, the BPI3Vc backbone further comprises an additional antigen coding sequence for at least one immunological active component against another disease-causing organism.

The entire teachings and content of the following references are incorporated by reference herein.

1. Chan, J. F.-W., S. Yuan, K.-H. Kok, K. K.-W. To, H. Chu, J. Yang, F. Xing, J. Liu, C. C.-Y. Yip, R. W.-S. Poon, H.-W. Tsoi, S. K.-F. Lo, K.-H. Chan, V. K.-M. Poon, W.-M. Chan, J. D. Ip, J.-P. Cai, V. C.-C. Cheng, H. Chen, C. K.-M. Hui, and K.-Y. Yuen. 2020. A familial cluster of pneumonia associated with the 2019 novel coronavirus indicating person-to-person transmission: a study of a family cluster. The Lancet 395:514-523.

2. Huang, C., Y. Wang, X. Li, L. Ren, J. Zhao, Y. Hu, L. Zhang, G. Fan, J. Xu, X. Gu, Z. Cheng, T. Yu, J. Xia, Y. Wei, W. Wu, X. Xie, W. Yin, H. Li, M. Liu, Y. Xiao, H. Gao, L. Guo, J. Xie, G. Wang, R. Jiang, Z. Gao, Q. Jin, J. Wang, and B. Cao. 2020. Clinical features of patients infected with 2019 novel coronavirus in Wuhan, China. The Lancet 395:497 506.

3. Wrapp, D., N. Wang, K. S. Corbett, J. A. Goldsmith, C.-L. Hsieh, O. Abiona, B. S. Graham, and J. S. McLellan. 2020. Cryo-EM structure of the 2019 nCoV spike in the prefusion conformation. Science 367:1260.

4. Stone, G. W., S. Barzee, V. Snarsky, K. Kee, C. A. Spina, X.-F. Yu, and R. S. Kornbluth. 2006. Multimeric Soluble CD40 Ligand and GITR Ligand as Adjuvants for Human Immunodeficiency Virus DNA Vaccines. Journal of Virology 80:1762.

5. Chen, X., J. L. Zaro, and W.-C. Shen. 2013. Fusion protein linkers: property, design and functionality. Adv Drug Deliv Rev 65:1357-1369.

6. Liang, B., J. O. Ngwuta, R. Herbert, J. Swerczek, D. W. Dorward, E. Amaro-Carambot, N. Mackow, B. Kabatova, M. Lingemann, S. Surman, L. Yang, M. Chen, S. M. Moin, A. Kumar, J. S. McLellan, P. D. Kwong, B. S. Graham, A. Schaap-Nutt, P. L. Collins, and S. Munir. 2016. Packaging and Prefusion Stabilization Separately and Additively Increase the Quantity and Quality of Respiratory Syncytial Virus (RSV)-Neutralizing Antibodies Induced by an RSV Fusion Protein Expressed by a Parainfluenza Virus Vector. Journal of virology 90:10022-10038.

7. Kim, J. H., S.-R. Lee, L.-H. Li, H.-J. Park, J.-H. Park, K. Y. Lee, M.-K. Kim, B. A. Shin, and S.-Y. Choi. 2011. High Cleavage Efficiency of a 2A Peptide Derived from Porcine Teschovirus-1 in Human Cell Lines, Zebrafish and Mice. PLOS ONE 6:e18556.

8. Edwards, C. P., and A. Aruffo. 1993. Current applications of COS cell based transient expression systems. Current Opinion in Biotechnology 4:558-563.

9. Liang, B., S. Munir, E. Amaro-Carambot, S. Surman, N. Mackow, L. Yang, U. J. Buchholz, P. L. Collins, and A. Schaap-Nutt. 2014. Chimeric Bovine/Human Parainfluenza Virus Type 3 Expressing Respiratory Syncytial Virus (RSV) F Glycoprotein: Effect of Insert Position on Expression, Replication, Immunogenicity, Stability, and Protection against RSV Infection. Journal of Virology 88:4237-4250.

10. Haller, A. A., M. MacPhail, M. Mitiku, and R. S. Tang. 2001. A Single Amino Acid Substitution in the Viral Polymerase Creates a Temperature-Sensitive and Attenuated Recombinant Bovine Parainfluenza Virus Type 3. Virology 288:342-350.

11. Bryson, D. G., B. M. Adair, M. S. McNulty, M. McAliskey, H. E. Bradford, G. M. Allan, R. T. Evans, and F. Forster. 1999. Studies on the efficacy of intranasal vaccination for the prevention of experimentally induced parainfluenza type 3 virus pneumonia in calves. The Veterinary record 145:33-39.

12. Haller, A. A., M. Mitiku, and M. MacPhail. 2003. Bovine parainfluenza virus type 3 (PIV3) expressing the respiratory syncytial virus (RSV) attachment and fusion proteins protects hamsters from challenge with human PIV3 and RSV. The Journal of general virology 84:2153-2162.

1-64. (canceled)

65. A composition comprising:
at least one SARS-CoV-2 RBD protein or at least one BPI3Vc-vectored SARS-CoV-2 RBD protein; and
a pharmaceutical-acceptable carrier selected from the group consisting of a solvent, a dispersion media, a coating, a stabilizing agent, a diluent, a preservative, an antimicrobial agent, an antifungal agent, an isotonic agent, and an adsorption delaying agent.

66. The composition of claim 65, wherein said protein has at least 90% sequence homology with SEQ ID NO. 3.

67. The composition of claim 65, wherein said protein is present in the final composition in an amount from 0.2 to about 400 μg/ml.

68. The composition of claim 65, further comprising an immune stimulant and/or at least one immunological active component against another disease-causing organism.

69. The composition of claim 65, wherein said BPI3Vc-vectored SARS-CoV-2 RBD protein is encoded by a BPI3Vc vector having a sequence with at least 90% sequence homology with SEQ ID NO. 13.

70. A composition comprising:
at least one SARS-CoV-2 nucleotide sequence selected from the group consisting of the nucleotide sequence encoding the RBD of SARS-CoV-2; and
a pharmaceutical-acceptable carrier selected from the group consisting of a solvent, a dispersion media, a coating, a stabilizing agent, a diluent, a preservative, an antibacterial agent, an antifungal agent, an isotonic agent, and an adsorption delaying agent.

71. The composition of claim 70, wherein said nucleotide sequence encodes a sequence having at least 90% sequence homology with either SEQ ID NO. 1 or SEQ ID NO. 3.

72. The composition of claim 70, wherein said nucleotide sequence is present in the final composition in an amount from 0.2 to about 400 μg/ml.

73. The composition of claim 70, further comprising an immune stimulant and/or at least one immunological active component against another disease-causing organism.

74. A method of inducing an immunological response against SARS-CoV-2 and/or reducing the incidence of, severity of, or transmissibility of SARS-CoV-2 comprising the steps of:
administering a composition selected from the group consisting of the composition of claim 65, the composition of claim 70, or a combination thereof, to an animal in need thereof.

75. The method of claim 74, wherein the animal in need thereof is selected from the group consisting of humans, dogs, cats, ferrets, bats, cattle, camels, hamsters, horses, chimps, gorillas, anteaters, dolphins, alligators, and sheep.

76. The method of claim 74, wherein said composition is administered a first time and a second time.

77. The method of claim 74, wherein said protein is present in the final composition in an amount from 0.2 to about 400 μg/ml.

78. The method of claim 74, further comprising an immune stimulant and/or at least one immunological active component against another disease-causing organism.

79. The method of claim 74, wherein the incidence of, severity of, or transmissibility of SARS-CoV-2 is reduced by at least 50%.

80. A composition comprising:
a vector comprising a BPI3Vc backbone and an inserted sequence, wherein said inserted sequence includes a SARS-CoV-2 RBD coding sequence.

81. The composition of claim 80, wherein said BPI3Vc backbone comprises a sequence having at least 90% sequence homology with SEQ ID NO. 13.

82. The composition of claim 80, further comprising a pharmaceutical-acceptable carrier selected from the group consisting of a solvent, a dispersion media, a coating, a stabilizing agent, a diluent, a preservative, an antibacterial agent, an antifungal agent, an isotonic agent, and an adsorption delaying agent.

83. The composition of claim 80, wherein said SARS-CoV-2 RBD coding sequence encodes a sequence having at least 90% sequence homology with SEQ ID NO. 3.

84. The composition of claim 80, wherein said BPI3Vc backbone further comprises an additional antigen coding sequence for at least one immunological active component against another disease-causing organism.
